(19) United States
(12) Patent Application Publication
(10) Pub. No.: US 2025/0084371 A1
(43) Pub. Date: Mar. 13, 2025

(54) CELL MONOLAYER BLOCKING AGENTS AND USES THEREOF

(71) Applicant: LUMICKS CA HOLDING B.V., Amsterdam (NL)

(72) Inventors: Domenico FASCI, Amsterdam (NL); Marinus Theodorus Johannes VAN LOENHOUT, Amsterdam (NL); Trillian Ashley GREGG, Amsterdam (NL); Serena Alba DAVOLI, Amsterdam (NL); Mattijs DE GROOT, Amsterdam (NL)

(21) Appl. No.: 18/725,497

(22) PCT Filed: Dec. 29, 2022
(86) PCT No.: PCT/EP2022/087997

§ 371 Date: Jun. 28, 2024

(30) Foreign Application Priority Data
Dec. 31, 2021 (NL) 2030377
May 13, 2022 (EP) 22173286.0
Jul. 20, 2022 (EP) 22186062.0

(51) Int. Cl. C12N 5/071; C07K 14/78

(52) U.S. Cl. CPC C12N 5/0602; C07K 14/78; C12N 2502/1114; C12N 2510/00

(57) ABSTRACT

The current invention relates to cell-cell interaction and in particular to cellular avidity. Provided are improved means and methods to study cell-cell interaction and characterizing cellular avidity. When target cells were prepared on a coated surface, and subsequently allowed to interact with cells, background binding can be observed. The current inventors sought to improve, i.e. reduce, background binding and found highly advantages blocking compounds that are an improvement over blocking compounds commonly used.

### INTRODUCTION

In the art, binding studies have been conducted with target cells being attached to a plate, e,g. a glass plate, wherein subsequent cells are added to the plate to interact (i.e. bind) with the attached target cells. In particular, cellular avidity experiments have been conducted in which target cells attached to a glass surface are to interact with subsequent cells, e.g. effector cells. However, a problem with target cells attached to a flat surface, wherein said surface has been treated with agents such as e.g. poly-L-lysine or the like to promote adhesion of the target cells to the surface, is that in addition to specific binding of the subsequent cells to the target cells, aspecific binding of the cells to the agent can occur.

Aspecific binding can occur due to many reasons, one of which being that the agent that has been used to coat a surface to which cells are to adhere, may be available for binding to the subsequent cells provided. Hence, in the art, standard blocking agents have been utilized such as for example serum, or the like. However, it was observed that such blocking agents may not sufficiently avoid binding of cells to coated surfaces. Without being bound by theory, this may be because serum or the like may in itself have aspecific binding properties, or such agents are not or less effective in binding with the coated surface, thereby still providing a coated surface to which cells can bind.

Hence, there is a need in the art to provide for means and methods that can reduce aspecific binding.

### SUMMARY OF THE INVENTION

When monolayers of target cells were prepared on a coated surface, and subsequently allowed to interact with control cells or effector cells with a receptor specific to the target cells, it was observed that a large portion of control cells appeared to bind to the target cells. This means that a large portion of the effector cells that bind to target cells can be attributed to aspecific binding, which is undesirable when assessing e.g. cellular avidity (see FIG. 1). The current inventors sought to improve, i.e. reduce, possible aspecific binding, using standard approaches known in the art. However, utilizing standard approaches like blocking with serum or the like, did not provide for a sufficient reduction of apparent aspecific binding, as observed during assessment of cellular avidity (not shown).

Hence, an object of the current invention is to provide for improved means and methods to reduce background binding to target cells attached to a coated surface that may be due to the coating, i.e. polypeptide coating. Provided herein are succinimidyl agents which can advantageously be used to reduce background binding. Such succinimidyl agents, including succinimidyl polyethylene glycols, as described herein and as shown in the examples, can react with the amine groups present in the polypeptide coating. Without being bound by theory, any coating that may have been available at the polypeptide coated surface with target cells attached thereto is reactively and/or sterically blocked from interaction with the subsequent cells provided to interact with the target cells. Polypeptides that may be contemplated as a coating, include, as shown in the examples, poly-L-lysin and fibronectin, but may not be limited thereto. As shown in the examples, in instances wherein target cells attached to a surface were treated with a succinimidyl PEG compound, a large reduction in binding of control cells to the target cells was observed, as compared with an only minor effect on specific binding of cells to the target cells, thereby resulting in a highly advantageous improvement of the so called “negative/positive control window”.

### DESCRIPTION OF THE FIGURES

FIG. 1. High aspecific binding of control cells in avidity experiments reduces the negative/positive window: Untransduced (UNT) or FMC63-transduced Jurkat (CAR) were layered on Z-Movi® chips coated with Poly-L-lysine (PLL), seeded with NALM6 (top panel) or Raji cells (lower panel) and avidity was measured. Plotted in grey scale are the percentage of bound cells at the y-axis (0-100%) and the force at the x-axis (0-1000 pN). The left side of the figure displays bar graphs indicating the percentage of bound effector cells at the end of the force ramp (1000 pN). A: UNT+NALM6, 11.5% of cells bound at 1000 pN, B: CAR+NALM6, 84.5% of cells bound at 1000 pN, C: UNT+Raji, 59.2% of cells bound at 1000 pN, D: CAR+Raji, 78.5% of cells bound at 1000 pN. The data represents mean±SD, n=2, and is representative of three independent experiments.

FIG. 2. Structure of the NHS-based blockers used and chemical reaction: A) NHS ester reaction scheme for chemical conjugation to a primary amine. R represents e.g. PEG or one end of a blocker/reagent having the NHS ester reactive group; P represents a polypeptide or other molecule that contains the target functional group (i.e., primary amine). B) Chemical structure of the BS(PEG)₅. C) Chemical structure of the MS(PEG)₄.

FIG. 3. Scheme of PLL and Fibronectin coated glass with amine groups.

FIG. 4. Blocking of PLL-coated chips with BS(PEG)₅ reduces binding of control cells: Chips were coated with PLL and incubated with different blocking agents as described in the examples. After the blocking step, Jurkat cells were introduced in the chips and incubated to allow binding. The avidity of the Jurkat cells to the plate was subsequently determined. Plotted in grey scale and as indicated with A-H) is the percentage of bound cells with the different agents at the y-axis (0-100%) and the force at the x-axis (0-1000 pN) (A, serum free RPMI medium; B, 1 mM BS(PEG)₅; C, 5 mM BS(PEG)₅; D, 20% fetal bovine serum (FBS); E, 50% FBS; F, 50% human serum (HS); G, 10% bovine serum albumin (BSA); and H, NALM6 condition medium). Using serum free RPMI medium (A), 93% of the cells were bound at the end (1000 pN) of the avidity measurement, at the other end of the spectrum, 5 mM BS(PEG)₅ (C) resulted in only 1.2% of cells to remain bound. The further agents resulted in binding of Jurkat cells at the end of the measurement in the range from 50%-90% (from high to low: 1 mM BS(PEG)₅ (B), 50% FBS (E), 20% (FBS) (D), NALM6 condition medium (H), 50% HS (F), 10% bovine serum albumin (BSA) (G), having percentages of 92.9%, 86.7%, 85.5%, 73.8%, 73.7%, and 51.5%, respectively).

FIG. 5. Blocking of human fibronectin-coated chips with BS(PEG)₅ reduces the binding of control cells: Chips were coated with human fibronectin and incubated with different blocking agents as described in the examples. After the blocking step, Jurkat cells were introduced in the chips and incubated to allow binding. The avidity of the Jurkat cells to the plate was subsequently determined. Plotted in the bar graph and as indicated with A-G is the percentage of bound effector cells at the end of the force ramp (1000 pN) with the different agents (A, serum free RPMI medium; B, 1 mM BS(PEG)₅; C, 5 mM BS(PEG)₅; D, 50% FBS; E, 50% HS; F, 10% BSA; G, NALM6 condition medium). Using serum free RPMI medium (A), 92.5% of the cells were bound at the end (1000 pN) of the avidity measurement, at the other end of the spectrum, 5 mM BS(PEG)₅ (C) resulted in only 43.2% of cells to remain bound. The further agents resulted in binding of Jurkat cells at the end of the measurement in the range from 50%-85% (from high to low: 50% HS (E), 10% BSA (F), 50% FBS (D), NALM6 condition medium (G), 1 mM BS(PEG)₅ (B), having percentages of 81%, 80.7%, 80.4%, 78.5%, 52.8%, respectively).

FIG. 6A. Blocking Raji seeded PLL-coated chips with NHS-based blockers reduces the aspecific binding of Jurkat control cells in avidity experiments: Chips coated with PLL and seeded with Raji cells were left untreated (DMSO vehicle) or treated with 4.5 mM BS(PEG)₅ or 5 mM MS(PEG)₄. The avidity experiment was then performed with untransduced (UNT) or FMC63-transduced primary T cells (CAR). In the bar graph is plotted the percentage of effector cells bound to the target cells at the end of the force ramp (1000 pN) where A: UNT+Raji, 68.7%, B: CAR+Raji, 90.9%, C: UNT+Raji+BS(PEG)₅, 15.6%, D: CAR+Raji+BS(PEG)₅, 78.3%, E: UNT+Raji+MS(PEG)₄, 32.8%, F: CAR+Raji+MS(PEG)₄, 84.7%. The blocking of the Raji seeded chips with BS(PEG)₅ and MS(PEG)₄ resulted in a difference in the positive/negative window of 62.7% and 51.9% respectively, while the window of the non-blocked sample was 22.2%. The data represents mean±SD, n=2 and is representative of two independent experiments.

FIG. 6B. Blocking of Raji seeded PLL-coated chips with NHS-based blocking agents does not reduce monolayer viability: Chips coated with PLL and seeded with Raji cells were left untreated (DMSO vehicle) (A, chip #1372) or treated with 4.5 mM BS(PEG)₅ (B, chip #1446) or 5 mM MS(PEG)₄ (C, chip #1381). The monolayer integrity was evaluated. No differences in target cell monolayer confluency, cell morphology or staining could be detected indicating the target cells tolerated the procedure well.

FIG. 7A. Blocking Raji seeded PLL-coated chips with NHS-based blockers reduces the aspecific binding of primary control T cells in avidity experiments: Chips coated with PLL and seeded with Raji cells were left untreated (DMSO vehicle) or treated with A) 4.5 mM BS(PEG)₅ or B) MS(PEG)₄. The avidity experiments were then performed with untransduced (UNT) or FMC63-transduced primary T cells (CAR). The avidity curves on the left side of the panel displays the percentage of bound cells under the different condition at the y-axis (0-100%) and the force at the x-axis (0-1000 pN). The bar graphs on the right side of the figure show the percentage of effector cells bound to the target cells at the end of the force ramp (1000 pN) where A: UNT+Raji, 64.7%, B: CAR+Raji, 72.7%, C: UNT+Raji+BS(PEG)₅, 39.9%, D: CAR+Raji+BS(PEG)₅, 66.3%. The data represents mean±SD, n=2 and is representative of two independent experiments.

FIG. 7B. Same experiment as FIG. 7A repeated with n=3: A: UNT+Raji, 78.3%, B: CAR+Raji, 71%, C: UNT+Raji+MS(PEG)₄, 42.6%, D: G: CAR+Raji+MS(PEG)₄, 67.8%. The data represents mean±SD, n=3 and is representative of three independent experiments. In both set of experiments the positive/negative window improved when the blocking reaction has been carried out.

FIG. 8A. Blocking PLL coated, Hela-CD19 seeded chips with NHS-based blockers reduces the aspecific binding of Jurkat control cells in avidity experiments: Chips coated with PLL and seeded with Hela-CD19 cells were left untreated (DMSO vehicle) or treated with 5 mM MS(PEG)₄. The avidity experiment was then performed with untransduced (UNT) or FMC63-transduced Jurkat (CAR). The avidity curve on the left side of the panel displays the percentage of bound cells under the different condition at the y-axis (0-100%) and the force at the x-axis (0-1000 pN). The bar graph on the right side of the figure shows the percentage of effector cells bound to the target cells at the end of the force ramp (1000 pN) where A: UNT+Raji, 33.4%, B: CAR+Raji, 82.8%, C: UNT+Raji+MS(PEG)₄, 5%, D: G: CAR+Raji+MS(PEG)₄, 68%. The measurement after the blocking reaction gave a pos/neg window of 63% while the control measurement gave a window of 49.4%. The data represents mean±SD, n=2.

FIG. 8B. Blocking fibronectin coated, Hela-CD19 seeded chips with NHS-based blockers reduces the aspecific binding of Jurkat control cells in avidity experiments: Chips coated with human fibronectin and seeded with Hela-CD19 cells were left untreated (DMSO vehicle) or treated with 5 mM MS(PEG)₄. The avidity experiment was then performed with untransduced (UNT) or FMC63-transduced Jurkat (CAR). The avidity curve on the left side of the panel displays the percentage of bound cells under the different condition at the y-axis (0-100%) and the force at the x-axis (0-1000 pN). The bar graph on the right side of the figure show the percentage of effector cells bound to the target cells at the end of the force ramp (1000 pN) where A: UNT+Raji, 56.9%, B: CAR+Raji, 90.2%, C: UNT+Raji+MS(PEG)₄, 16.8%, D: CAR+Raji+MS(PEG)₄, 70.1%. The measurement after the blocking reaction gave a pos/neg window of 53.3% while the control measurement gave a window of 33.3%. The data represents mean±SD, n=2.

FIG. 9. Blocking of target Raji cells does not affect Jurkat-CAR activation: Raji cells were left untreated (DMSO vehicle) or treated with 5 mM BS(PEG)₅ or 5 mM MS(PEG)₄ for 15 minutes and co-incubated for 4 h at 1:1 ratio with untransduced (UNT) or FMC63-transduced Jurkat (CAR). T cell activation was then assessed by staining for the CD69 activation marker. The bar plot displays the percentage of CD69 positive cells measured in the samples analyzed. A: UNT, 0.22%, B: UNT+Raji, 0.8%, C: UNT+Raji+BS(PEG)₅, 0.6%, D: UNT+Raji+MS(PEG)₄, 0.8%, E: CAR, 1.1%, F: CAR+Raji, 24.7%, G: CAR+Raji+BS(PEG)₅, 30%, H: CAR+Raji+MS(PEG)₄, 27%. The data represents mean±SD, n=2 and is representative of two independent experiments.

FIG. 10. Blocking of target Raji cells does not affect primary CAR-T cell activation: Raji cells were left untreated (DMSO vehicle) or treated with 4.5 mM BS(PEG)₅ for 15 minutes and co-incubated for 7 h at 1:1 ratio with untransduced (UNT) or FMC63-transduced primary T cells (CAR). T cell activation was then assessed by measuring the IFNγ levels from the co-culture supernatants. The bar plot displays the amount of IFNγ measured in the samples analyzed. A: UNT, 0 pg/mL, B: UNT+Raji, 0 pg/mL, C: UNT+Raji+BS(PEG)₅, 11 pg/mL, D: CAR, 0 pg/mL, E: CAR+Raji, 1479 pg/mL, F: CAR+Raji+BS(PEG)₅, 1633 pg/mL. The data represents mean±SD, n=2, and is representative of two independent experiments.

FIG. 11. Schematic showing target cells and cells of interest and cellular avidity. Target cells (2) are provided on a surface (1), which as depicted is in this case a flat surface. The target cell expresses ligands and receptors, likewise the cell (6) to be targeting the target expresses ligands and receptors as well (3). A specific ligand-receptor interaction (4 and 5) can be the driving force for the forming of a cell-cell bond with multiple ligand-receptor interactions combined resulting in strong cell-cell binding. To rupture this cell-cell bond, a force (7) is exerted on the cell (6) away from the target cell (2), which can be in the z-axis direction e.g. when the flat surface is defined as being in the x-y plane. Alternatively, this can also be in the x or y-axis direction. When the cell-cell bond is ruptured, the cell moves away from the cell surface and/or the target cell and this event can be detected and/or detached cells can be collected and quantified and/or further analyzed.

FIG. 12. In a cellular avidity measurement with cells attached to a surface (depicted as grey cells), cells (depicted as white cells) are interacted with the attached cells to bind therewith, e.g. utilizing target cells expressing an antigen and cells of interest such as effector cells with a CAR against the antigen, as depicted in a). After a defined incubation, a force, Fₘ applied away from the attached cells. This results in cells detaching therefrom, either because they did not bind to the cells attached to the surface or because the binding strength was not strong enough, e.g. when aspecifically bound. Cells that remain were sufficiently strongly bound to the attached cells as depicted in b). Such a scenario is e.g. employed in the examples as described herein. Cells that formed e.g. a synapse may substantially remain (indicated with hashes). Cells may also be resuspended and have a differential force, Fₙ, is applied. Such an applied differential force may be larger than typically used when cells are attached (Fₘ). This differential force can also break cell-cell bonds, preferably aspecific cell-cell bonds as these bonds may be less strong when compared with specific cell-cell bonds such as e.g. synapse bonds, thereby resulting in e.g. substantially specific cell-cell bonds. The cell suspension may be subsequently analysed with regard to singlets (either white or grey cells) and doublets (grey cell bound with white cell), which numbers may be used, e.g. to provide a cellular avidity score. In accordance with the invention, one may apply a differential force (shown in c), after a force has been applied, e.g. away from attached cells (in the scenario obtained as shown in b), or one may apply a differential force after incubation on attached cells (in the scenario as obtained in a)). Cellular avidity scores may be determined by determining the number of grey cells and white cells bound to each other (doublets) and/or by counting the number of grey cells bound to white cells, e.g. effector cells bound to target cells, that formed a specific bond such as a synapse (doublets with hashes) (depicted in d). Cellular avidity scores can be calculated taking into account initial amount of cells provided and/or single cells obtained in the methods. For example, by calculating the ratio of the number of cells of interest that remained bound to target cells after exerting the force to the number of cells of interest initially provided.

FIG. 13. Chips coated with PLL were seeded with Raji cells in PBS and left untreated (standard seeding, black line (i.e. middle line in graph)) or treated with 10 mM BS(PEG)₉ (covalent monolayer—dark grey line (i.e. upper line in graph)) or a chip coated with PLL was reacted with 5 mM MS(PEG)₄ before seeding the Raji (negative control—light grey line (i.e. lower line in graph)). Each chip was subsequently subjected to 4 acoustic force ramps from 1 to 1000 pN in 2.5 minutes and the monolayer confluency was assessed with the Oceon® software. The graph at the lower end of the figure indicates the relative change in monolayer confluency after force application for each condition. It can be concluded that reacting Raji seeded PLL-coated chips with BS(PEG)₉ improves the stability of the target cell monolayer to acoustic force application.

FIG. 14. Chips coated with PLL and seeded with Raji cells were left untreated (standard seeding—top panel) or treated with 10 mM BS(PEG)₉ (covalent monolayer seeding—bottom panel). The avidity experiments were then performed with untransduced (UNT) or FMC63-transduced primary T cells (CAR). The avidity curves on the left side of the figure display the percentage of bound cells under the different conditions at the y-axis (0-100%) and the force at the x-axis (0-1000 pN). The bar graphs on the right side of the figure show the percentage of effector cells bound to the target cells at the end of the force ramp (1000 pN) where A: CAR+Raji, 85.6%, B: UNT+Raji, 63.3%, C: CAR+Raji+BS(PEG)₉, 61.4%, D: UNT+Raji+BS(PEG)₉, 10.3%. The data represent mean±SD, n=2 and are representative of one experiment. It can be concluded that reacting Raji seeded PLL-coated chips with BS(PEG)₉ reduces the aspecific binding of primary control T cells and improves the positive/negative window in avidity experiments.

### DETAILED DESCRIPTION

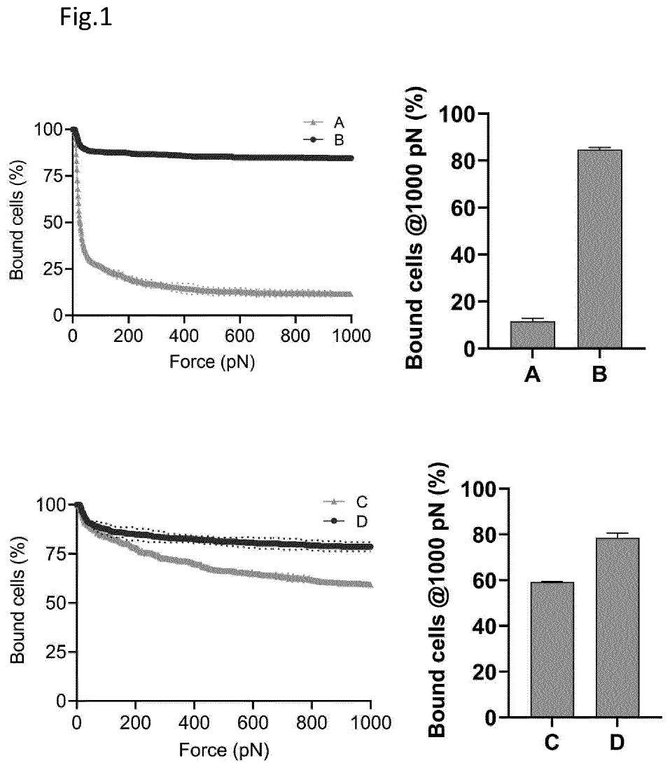

As said, when target cells are attached to a coated surface, and subsequently allowed to interact with control cells or cells of interest, e.g. with a receptor specific to the target cells, substantial background binding can confound specific cellular avidity, in particular in methods wherein cellular avidity is an important parameter. For example, the current inventors observed that the detachment of cells of interest and control cells, when applying a force thereon, sometimes results in a large portion of control cells to remain attached to the target cells (see e.g. FIG. 1, bottom panel). Hence, when studying the binding of cells of interest to target cells by exerting a force on the cells of interest, such substantial background binding may confound the assessment and/or use of (e.g. when selecting cells) specific cellular avidity of (candidate) cells of interest with target cells. This is because a large portion of the cells of interest that bind to target cells may be cells that bind aspecifically or at least cannot be differentiated therefrom, which is highly undesirable. Hence, the current inventors sought to improve, i.e. reduce, background binding in means and methods relying on cellular avidity.

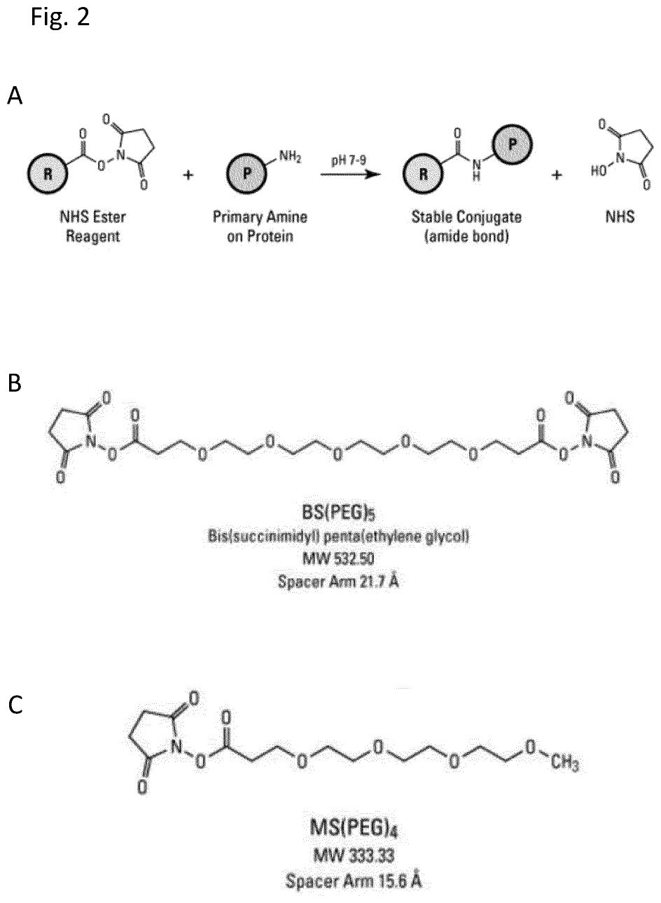

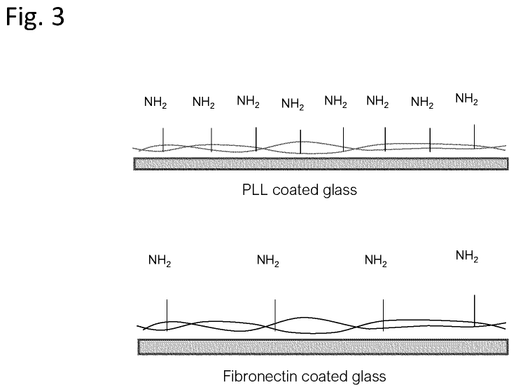

Hence, an object of the current invention is to provide for improved means and methods to reduce background binding to target cells attached to a coated surface that may be due to the coating used, i.e. a polypeptide coating. Surprisingly, it was found that NHS ester reagents, i.e. agents that can form a stable conjugate bond with primary amines (see FIG. 2A), are highly useful as blocking agents, i.e. agents that can reduce background binding. Without being bound by theory, such agents are to react with amine groups present in the coating agent, e.g. because the coating agent comprises a polypeptide, such that these amine groups of the coating agents no longer would be available to attach to the cells of interest. Surprisingly, as shown in the examples herein, using such NHS ester reagents can reduce background binding, while at the same time not substantially affecting cell viability and functionality. Hence, in one embodiment, a method is provided for preparing target cells attached to a coated surface, comprising the steps of:
providing target cells attached to a surface coated with a polypeptide;
providing an NHS ester reagent;
blocking the target cells attached to the coated surface with the NHS ester agent.

As said, target cells are first provided attached to a surface coated with a coating agent, which includes a polypeptide. The target cells can be any target cell which is of interest to study, for example utilizing means and methods relying on cellular avidity measurements such as shown in the example section. The surface is preferably suitable for microscopy methods or the like, and/or preferably suitable for applying a force. In any case, first the target cells are provided attached to a surface coated with a polypeptide.

Further, an NHS ester reagent is provided. In accordance with the invention, an NHS ester reagent is a compound comprising at least one succinimidyl group, which functional group is capable of forming an ester bond, e.g. with a primary amine group on polypeptides. Next, the target cells attached to the coated surface are blocked with the NHS ester agent, e.g. by providing a buffer suitable for the blocking reaction and sustaining cell viability of the target cells, e.g. comprised in a physiologically acceptable buffer. As the NHS ester reagent reacts readily with proteins, such a blocking buffer preferably does not comprise a substantial amount of protein as this may interfere with the blocking reaction. As shown in the example section, typical buffers include buffers such as serum free media and/or PBS. As cells may be grown in serum/protein containing culture media, as commonly known in the art, the target cells may be washed first with PBS or the like before the blocking reaction. Once the blocking reaction has been allowed to take place for a defined period, the NHS ester reagent is removed and the target cells may be washed with serum containing medium to stop the reaction. This way, target cells attached to a surface coated with a polypeptide can be provided that advantageously have reduced aspecific binding, e.g. due to coating agent being exposed and available for attaching to the cells of interest.

Of course, instead of providing already prepared target cells attached to a surface coated with a polypeptide, the means and methods in accordance with the invention also can comprise providing 1) target cells and 2) a surface coated with a polypeptide; or providing 1) target cells and 2) a surface, and 3) a composition for coating comprising a polypeptide, and the preparation of target cells being attached to a surface coated with a polypeptide.

Hence, in one embodiment, a method is provided for preparing target cells attached to a coated surface, comprising the steps of:
providing a surface coated with a polypeptide for attachment of target cells;
providing target cells;
providing an NHS ester reagent;
contacting the target cells with the coated surface and allowing the target cells to attach to the coated surface;
blocking the coated surface with attached target cells with the NHS ester agent.

In another embodiment, a method is provided for preparing target cells attached to a coated surface, comprising the steps of:
providing a surface for attachment of target cells;
providing target cells;
provide a polypeptide for coating;
providing an NHS ester reagent;
coating the surface with a polypeptide to provide a coated surface;
contacting the target cells on the coated surface and allowing the target cells to attach to the coated surface;
blocking the target cells attached to the coated surface with the NHS ester agent.

As said, without being bound by theory, the NHS reagent provided, e.g. in a suitable buffer, is to react with the coating agent that was used to attach the target cells to the surface. The coating agent is to comprise a polypeptide, and the NHS ester reagent can react with the primary amine groups comprised therein, i.e. such as comprised in lysine residues (K, Lys). NHS esters are known in the art, and it may be contemplated to utilize other agents equally capable of reacting with amine groups as an alternative. For example, these may include isothiocyanates, isocyanates, acyl azides, sulfonyl chlorides, aldehydes, glyoxals, epoxides, oxiranes, carbonates, aryl halides, imidoesters, carbodiimides, anhydrides, and fluorophenyl esters. Most of these groups conjugate to amines by either acylation or alkylation. Without being bound by theory, as long as the agent is capable of reacting with the coating agent, e.g. with the amine groups, thereby not substantially interfering with the positive sample binding and reducing aspecific binding, as shown in the examples, such an agent can be contemplated.

However, it is preferred to use an NHS ester in accordance with the invention. NHS esters are known in the art and include reactive groups formed by carbodiimide-activation of carboxylate molecules. These agents may be referred to in the art as crosslinkers, as these can form a link between two molecules (e.g. between a polypeptide and a PEG molecule, and off course when an agent has two reactive groups, two polypeptides may be joined: polypeptide 1—link—PEG—link polypeptide 2). As these agents are used herein as blocking agents, reference herein is made as such. In accordance with the invention the NHS ester, or the like, is to react with primary amines to block potential attachment of cells of interest thereto. NHS ester reagents react with primary amines in physiologic to slightly alkaline conditions (pH 7.2 to 9) to yield stable amide bonds. The reaction releases N-hydroxysuccinimide (NHS). Hence, NHS ester reagents are highly useful under physiological conditions. The rate of hydrolysis increases with buffer pH and contributes to less-efficient blocking in less-concentrated protein solutions. NHS-ester blocking reactions are most commonly performed in phosphate, carbonate-bicarbonate, HEPES or borate buffers at pH 7.2 to 8.5 for 0.5 to 4 h at room temperature or 4° C. Primary amine buffers such as Tris (TBS) are not compatible, because they compete for reaction; however, as shown in the example section, it is useful to add Tris or glycine buffer at the end of a conjugation procedure to quench (stop) the reaction. In accordance with the invention, NHS ester reagents are contemplated that result in an amide bond as shown FIG. 2A.

Preferably, the NHS ester reagent comprises a spacer, e.g. a polymer of a relative short length, up to about 100 Angstrom. Such a polymer preferably is inert. Preferably, the polymer is a polyethylene glycol (PEG). More preferably, the polyethelyne glycol length is up to 25 ethylene glycol units in length. Preferably, the number of units is selected from the range of 2 to 25, or from 3 to 10. Hence, in a further embodiment, the NHS ester is a PEG-NHS ester. The polymer may have at each end an NHS group or have at one end an NHS group, such may be referred to as bis(succinimidyl) (BS) and mono(succinimidyl) (MS), respectively. Hence, in another embodiment the NHS ester reagent is a BS or MS polymer. In yet another embodiment, the BS and MS polymer is an BS(PEG)n or MS(PEG)n, wherein n represents the number of ethylene glycol units, which, as said, preferably is up to 25 in length. In another embodiment, the NHS ester reagent is selected from BS(PEG)n, and MS(PEG)n, wherein n is selected from 2, 3, 4, 5, 6, 7, 8, 9, 10, 11, 12, 13, 14, 15, 16, 17, 18, 19, 20, 21, 22, 23, 24, and 25. In yet a further embodiment, n is selected from 2, 3, 4, 5, 6, 7, 8. In yet another further embodiment, n is selected from 4 and 5. In any case, the polymer length is selected and/or type thereof, such that aspecific binding, or binding of control cells which may be attributed to e.g. binding to the coating peptide which may be available for attaching cells, is reduced, while specific binding, is substantially retained.

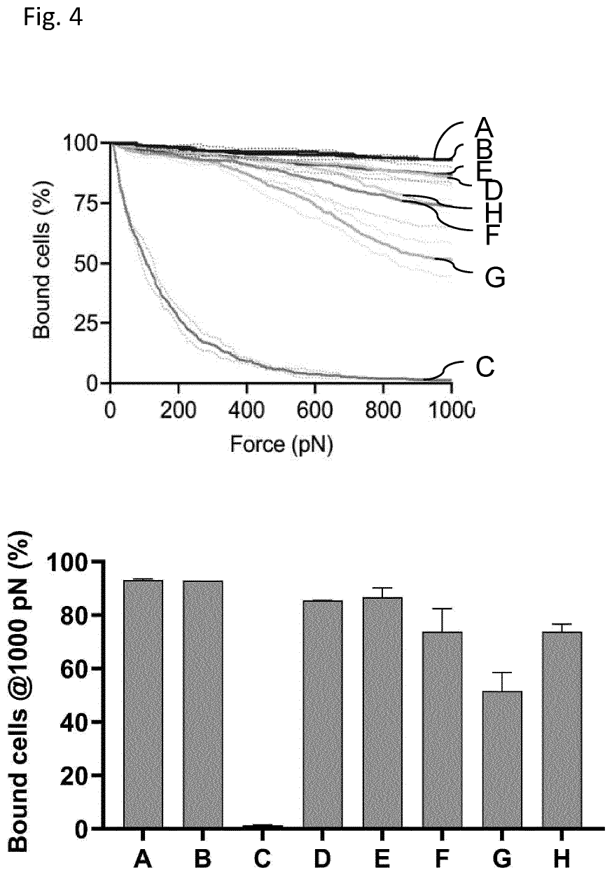

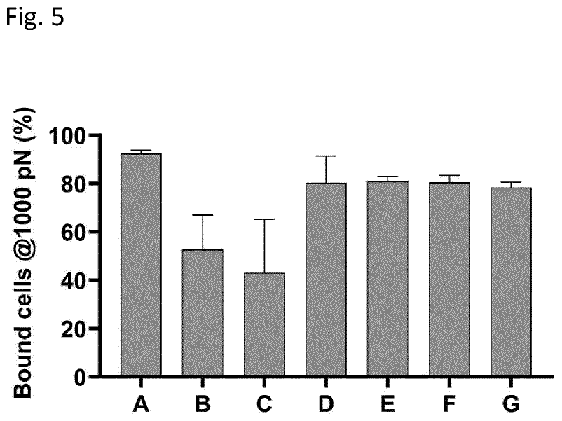

With regard to the amount of NHS ester reagent, or the like, used, this can be established by testing different concentrations and/or incubation times. In one embodiment, the incubation of cells is up to 30 minutes, for example for about 5, about, 10, about 15, about 20, about 25 or about 30 minutes. In another embodiment, the blocking reaction is about 10 to 20 minutes. Of course, the length of the reaction required to achieve sufficient blocking may depend on the concentration of the reagent used. Hence, in a further embodiment, the concentration of the NHS ester reagent can be up to 15 mM. The length of the reaction and the concentration of NHS ester reagent can also depend on the type of coating agent that was used. For example, as shown in the example section, the coating agent poly-L lysine has many amine groups, hence, a 1 mM concentration was not sufficient and 5 mM was shown to be more effective (see FIG. 4), as compared with fibronectin coating, wherein 1 mM and 5 mM concentrations were shown to have a similar effect (see FIG. 5). The reaction can be easily controlled by quenching the reaction by e.g. changing the buffer to a TBS buffer, or the like, as shown e.g. in the example section. In any case, suitable concentrations and/or reaction times for efficient blocking can be well established for NHS esters.

As said, the coating preferably comprises a polypeptide. In a further embodiment, the coating comprises a polypeptide selected from the group consisting of fibronectin, poly-L-lysine, poly-D-lysine, poly-L-ornithine, laminin, collagen, fibronectin, fibrinogen, vitronectin, osteopontin thrombospondin, VEGF, VCAM-1, ICAM. In another further embodiment, the coating comprises one or more of the groups consisting of fibronectin, poly-L-lysine, poly-D-lysine, poly-L-ornithine, laminin, collagen, fibronectin, fibrinogen, vitronectin, osteopontin thrombospondin, VEGF, VCAM-1, and ICAM. These polypeptides may attach to the surface and in their turn subsequently attach to the target cells thereby attaching the target cells to the surface. In any case, a suitable coating comprising a polypeptide may be selected such that the target cells that are attached to the surface allow for the target cells to remain attached to the surface when applying a force on the control cells or cells of interest. In other words, when a force is applied on the control cells or cells of interest which allows for these cells to detach from the target cells, the target cells are to substantially remain attached to the surface.

Furthermore, the surface may be any suitable surface, but may preferably be a glass surface, preferably a glass surface in a chip. Surfaces suitable for attachments of target cells utilizing a coating, such as polypeptide, are well known in the art. For example, a glass or plastic surface may be utilized. A surface material may be preferred which allows to detect for attachment to and/or detachment of the target cells, i.e. of cells of interest and/or control cells thereof. Such a surface material for instance also may allow for microscopy methods. As said, in order to attach cells to the surface, the surface is pretreated with a coating such as a polypeptide. The target cells that are attached to the surface, preferably are attached as a monolayer as this provides for better controllable conditions when applying the force on the control cells or cells of interest.

In any case, by applying the means and methods in accordance with the invention to thereby prepare target cells attached to a surface, target cells attached to a surface can be provided that are suitable in further means and methods for determining cellular avidity of provided cells, e.g. cells of interest and/or control cells thereof. As shown in the example section, when determining cellular avidity utilizing the z-Movi® device and chips, target cells are seeded in the chips in a small volume to attach to the coated surface, and a subsequent cellular avidity measurement is performed within hours, e.g. on the same day, after seeding. Hence, these circumstances may not be ideal for cell culture and/or attachment, and it may in particular be challenging to prepare target cells, e.g. a monolayer thereof, attached to a surface suitable for use in the z-Movi® device.

The target cells provided that are attached to the surface and blocked in accordance with the invention, preferably are attached as a monolayer. The monolayer preferably is at high confluency. The subsequent cells of interest (and control cells thereof) that are to interact with the target cells are preferably provided in a relatively low cell density as compared with the target cells, such that substantially all cells of interest (and control cells thereof) can interact with a target cells (there are more target cells per cell of interest). Such provides for advantageous controllable conditions when applying the force on the control cells or cells of interest.

Nevertheless, the means and methods in accordance with the invention find use in further methods as well which may suffer from aspecific binding to target cells, and the like. Hence, although in particular useful for the z-Movi® device, the means and methods in accordance with the invention are not limited to such a particular use.

Hence, in a further embodiment, a method is provided for determining cellular avidity comprising:
providing target cells attached to a coated surface in accordance with the invention;
providing cells of interest;
incubating the target cells with the provided cells of interest; and
determining the cellular binding avidity of the cells of interest with the target cells.

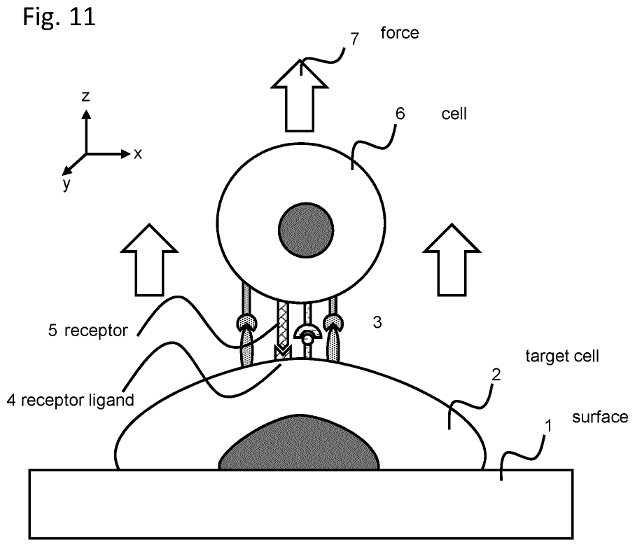

It is understood that “cellular avidity” as used throughout herein comprises the overall strength of interactions occurring in a cell to cell contact, involving a diversity of molecules at the surfaces of the cells that interact (see e.g. FIG. 11). Such interactions may include a diversity of receptor-ligand pairs, among which e.g. a specific receptor-ligand interaction, occurring at the membrane surface of a cell. For example, when a T-cell receptor triggers the formation of an immune synapse by recognizing an antigen presented by an MHC molecule at an antigen presenting cell, the synapse formation involves such multitude of interactions, as also other membrane bound molecules are involved in the interactions (such as integrins and the like). Hence, “cellular avidity” may not be restricted to the interaction of e.g. the alpha and beta chain of the TCR and the antigen presented by MHC, but rather involves a multitude of interactions working jointly forming a strong bond between e.g. cells. It may also involve active signalling and processes internal to the cells such as e.g. during immune synapse formation. It is understood that the cellular avidity of a cell of a certain type is defined relative to its target cell and conditions tested.

Hence, when performed such a method as described above, background binding can be substantially reduced. With regard to the cells that are provided, this may be any cells for which the cellular avidity towards the target cells is of interest to investigate. It is understood that in accordance with the invention target cells and cells of interest relate to two different cells which are to interact specifically with each other. Hence, one of the target cells and cells of interest expresses a ligand on its surface and the other cell expresses a receptor for that ligand. As control cells, the same cells as the cells of interest or target cells are utilized but these cells e.g. do not express such a ligand or receptor or express a variant thereof which is not functional. Hence, whereas as described herein reference can be made to cells of interest expressing a receptor, implying the target cells expressing a ligand, the means and methods in accordance with the invention herein throughout contemplate that cells of interest may express a ligand and target cells can express a receptor instead. Of particular interest and as further described below, in accordance with the invention, cells of interest (or, conversely target cells) can be T-cells or the like, which are to specifically target e.g. a cancer cell or other antigen presenting cell presenting a defined antigen on its surface.

In a further embodiment in a method for determining cellular avidity, control cells of the target cells or of the cells of interest are provided and the cellular avidity determined thereof as well. The control cells may be of target cells and of cells of interest. The method may be performed separately with control cells and cells of interest. When the control cells are of the cells of interest, the steps of the method are further performed by contacting the control cells and/or cells of interest with the target cells. Control cells and/or cells of interest that have detached and/or remained attached are determined and cellular avidity scores provided for the control cells and/or cells of interest with the target cells. Instead of providing control cells of the cells of interest, alternatively control cells of the target cells may be provided attached to a surface. In such a scenario, cells of interest are contacted with the target cells attached to a surface and subsequently, cells of interest are contacted with the control cells of the target cells attached to a surface, and in each case cells of interest that have detached and/or remained attached determined and cellular avidity scores provided for the cells of interest with the control cells of the target cells and of the target cells. These cellular avidity scores can be likewise compared and are of interest.

One may also perform the method in a combined fashion, e.g. when immobilized cells of interest and control cells are differentially labelled or can be otherwise distinguished from each other when determining cellular avidity. It is understood that in accordance with the invention target cells and cells of interest relate to two different cells which are to interact specifically with each other, e.g. one cell expressing a ligand on its surface whereas the other cell expresses a receptor for that ligand. As control cells, the same cells as the cells of interest can be utilized but these cells e.g. do not express such a ligand or receptor or express a variant thereof which is not functional or not specific for the counterpart present on the target cells. The target cells in accordance with the invention are defined to be the cells immobilized, i.e. attached to a surface. The cells of interest are defined not to be immobilized. It is understood that in accordance with the invention target cells and cells of interest relate to two different cells which are to interact specifically with each other. Which cell is immobilized may not be of importance for determining cellular avidity between the cells. As long as a force can be applied to the cells binding to the immobilized cells, and detachment and/or attachment of cells can be determined, determination of the cell numbers thereof allows one to determine cellular avidity. Hence, one of the target cells and cells of interest expresses a ligand on its surface and the other cell expresses a receptor for that ligand. As control cells, the same cells as the cells of interest or target cells can be utilized but these cells e.g. do not express such a ligand or receptor or express a variant thereof which is not functional. If the control cells are control cells of the target cells, these may be immobilized as well. If the control cells are control cells of the cells of interest, these may not be immobilized. Of particular interest and as further described below, in accordance with the invention, cells of interest (or, conversely target cells) can be T-cells or the like, which are to specifically target e.g. a cancer cell or other antigen presenting cell presenting a defined antigen on its surface. Hence, accordingly, in methods of the invention the target cells attached to the surface, i.e. the immobilized cells, may be cancer cells, and the cells of interest may be T-cells. Conversely, in methods of the invention, one may also choose to have the T-cell immobilized, as a target cell, and have the cancer cell as a cell of interest. The latter may complicate cell sorting of T-cells, but may be advantageous in scenarios where certain cancer cells are difficult to immobilize and/or different cancers cells may be studied. Of particular interest and as further described below, in accordance with the invention, cells of interest can be T-cells or the like, which are to specifically target e.g. a cancer cell or other antigen presenting cell presenting a defined antigen on its surface.

It is understood that in accordance with the invention, when e.g. and preferably, cellular avidity measurements are performed e.g. with control cells, cells of interest, and perhaps repeating cellular avidity measurements, this may involve utilizing the same target cells attached to a surface and/or may involve utilizing multiple provided target cells attached to a surface. For example, as shown in the examples, a chip (such as described i.a. in Fernández de Larrea, C., et al. (2020). Blood Cancer Discovery, 1(2), 146-154, WO2018083193, and such as available from LUMICKS for the z-Movi® device (see i.a. z-Movi-Brochure_2021.pdf, available from <https://lumicks.com/products/z-Movi-cell-interaction-studies/#brochure>) with target cells attached to a surface may be repeatedly used in the z-Movi® device and cells that have remained bound to the target cells and remain bound to the target cells in a subsequent measurement may e.g. be masked in analysis (e.g. by the software or by manually identifying these cells and excluding these in a subsequent measurement). Hence, it is understood that repeating the methods may be performed with the same target cells attached to a surface or may be performed with further provided target cells attached to a surface. When multiple surfaces with attached target cells are provided, it is understood that of course these are prepared in the same way such that different measurements with each of the multiple surfaces can be compared and are substantially reproducible. Hence, in one embodiment, the method for determining cellular avidity is repeated with the same target cells attached to a surface, e.g. as a monolayer.

In a preferred embodiment, the cellular binding avidity is determined by exerting a force on the provided cells of interest away from the target cells. It is understood that in this step, the force applied may be perpendicular (in the direction of z-axis) to the surface (x,y) to which the target cells are attached, for example when a centrifugal force or acoustic force is applied. The force may also be lateral (x-axis or y-axis), for example when a shear force is applied (see e.g. FIG. 11). In any case, the force is applied and is controlled such that a defined force is exerted on the cells that interact with the target cells. It is understood that the force that is exerted on the cells attached to the target cells is to be substantially equal, such can be achieved e.g. when using a flat surface as depicted in FIG. 11. Other suitable surface shapes may be used (e.g. a tube with exerted concentrical force or laminar flow force in the direction of the length of the tube), as long as the force exerted can be substantially equal at a defined surface area, such a surface shape may be contemplated. The force required to move a cell away from the target cell preferably can be detected, e.g. via microscopy or other means, to which may be referred to as a cell detachment event. This way, cell detachment events can be monitored and counted.

WO 2018/083193 discloses a method, system and sample holder for manipulating and/or investigating cellular bodies; it makes use of acoustic forces generated by ultrasound standing waves in a microfluidic flow-cell. The acoustic force allows application of force to thousands of cells in parallel, for example, to thousands of T-cells in contact with a monolayer of cancer target cells. By detecting cell-cell rupture events (e.g. (CAR-)T cells releasing from a tumor cell monolayer) as a function of the acoustic force and/or by application of fluid flow in relation to an applied acoustic force, cell-cell binding interactions may be efficiently analyzed and/or cells may be sorted according to cell-cell binding interactions. For example, an avidity curve can be generated by plotting the percentage of bound CAR T-cells as a function of the applied force (see e.g. FIG. 1). Such a system, which is commercially available from LUMICKS and known as z-Movi®, has been used in the example section as described herein.

However, the means and methods in accordance with the invention when exerting a force as disclosed herein are not restricted to the use of acoustic force, but are of use with other types of forces that can be applied on cells, such as centrifugal (or other acceleration) force or a shear flow force, and which can provide for similar graphs and plots as shown in the examples herein, all relying on the same inherent properties of cellular avidity. As long as a force can be applied and controlled on cells of interest that bind/interact with target cells attached to a surface such a force can be contemplated in accordance with the invention. Hence, in a further embodiment, the cellular avidity is determined by exerting an acoustic force, shear flow or a centrifugal force.

With regard to the cellular avidity, it is understood that this is to express the strength of binding of cells of interest (or control cells thereof) to the target cells. It is understood that where we refer to specific forces applied to cells this may refer to average forces, e.g. such forces may not be fully homogeneous, for example over the contact surface as may be the case with acoustic forces and shear-flow forces (see e.g. Nguyen, A., Brandt, M., Muenker, T. M., & Betz, T. (2021). Lab on a Chip, 21(10), 1929-1947 for a description of force inhomogeneities in acoustic force application)

Also for shear-flow forces the forces may also not be fully homogeneous, for example since the flow speed near the side walls of a flow channel (e.g. with a rectangular cross section) may be lower than in the center of the flow cell (due to the no-slip boundary condition). By choosing a cross section with a high aspect ratio (low and wide) these flow effects may be minimized such that only a few percent of the cells experience a substantially smaller force than the cells in the center of the flow cell. Other methods to mitigate such effects and to specifically select cells that have experienced similar forces may include using flow cell geometries with multiple fluid inlets and/or outlets such that the properties of laminar flow can be used to ensure cells of interest only land in regions of homogeneous force and/or are only selected from regions of homogeneous force. In one example, by using three channel inlets side by side one can use the side channels as sheath flow channels to focus cells of interest inserted into the center channel where the acoustic and/or shear force may be substantially homogeneous. The sheath flow fluid may be the same buffer fluid as is used for the sample cells but then free of sample cells. By increasing the flow speed through the sheath flow channels the cells are more focused and confined to the center of the channel while by reducing the sheath flow speed the cells are allowed to spread out more. Similarly, on the collection side flows in three side-by-side collection channels may be controlled to possibly discard cells flowing close to the channel boundaries and only collecting cells from the center of the channel. By controlling the relative flow speeds of such side channels and the center channel asymmetrically the location of the effective interaction region of the sorting device can be further controlled and cells that have underwent defined forces can be selected and/or detected.

Further means to enable collection of cells from a specific interaction region (and therefore collection of cells that experienced a defined force) include means and methods wherein cells of interest may be provided with a photoactivatable label which may be subsequently activated by illumination with light of a suitable wavelength only in a well-defined interaction region of the device (e.g. near the center of a flow channel or in a center region under an (acoustic) force transducer) to photoactivate and/or switch the dye. Subsequently, the cells can be sorted for example using fluorescence activated cell sorting (FACS) and only those cells which are activated are further used according to the methods described herein thereby obtaining the cells on which defined forces have been exerted. This may for example be highly useful for collecting cells that remained attached to the target cells. For example, the target cells and cells bound thereto may be trypsinized thereby obtaining both the target cells and cells that remained bound thereto in a suspension. Alternatively, attached cells can also be simply collected with physical means (e.g. scraping) from the area of interest, i.e. the surface area with a well-defined nominal force.

For centrifuge forces it is easier to ensure that the force applied is homogeneous across the whole interaction region since such a force does not depend strongly on a location on a surface with respect to a force transducer and/or the wall of a flow channel or sample holder.

Accordingly, in connection to the subject matter disclosed herein, means and methods exist which allows one to exert forces on cells attached to a surface and collect the cells, detached and/or attached cells, on which defined forces have been exerted.

It is understood that with regard to the force exerted, the exact forces experienced by cells may also depend on cell size and or other cell properties such as density and compressibility. The force may be a nominal force and not the true force experienced by the control cells or the cells of interest. E.g. it may be hard to precisely predict the average cell size, density, compressibility, etc. of the cells and the force may have been calculated based on theory alone or may have been calibrated using test particles with specific (preferably known) properties (see e.g. Kamsma, D., Creyghton, R., Sitters, G., Wuite, G. J. L., & Peterman, E. J. G. (2016). Tuning the Music: Acoustic Force Spectroscopy (AFS) 2.0. Methods, 105, 26-33). The force may be such a calculated or calibrated force expressed with units of N (e.g. pN) but it may also be expressed without calibration as the input power (Vpp) applied to a piezo element (see Sitters, G., Kamsma, D., Thalhammer, G., Ritsch-Marte, M., Peterman, E. J. G., & Wuite, G. J. L. (2014). Acoustic force spectroscopy. Nature Methods, 12(1), 47-50), as angular velocity squared (ω²) in the case of centrifugal force application or as flow speed v and or as shear stress (Pa) in applications using shear forces. As long as the forces exerted by the devices, e.g. shear force, acoustic force, or centrifugal forces, but not limited thereto, can be varied and controlled and reproduced in such devices such devices are suitable for the means and methods in accordance with the invention.

As the percentage of cells that remains bound at a certain applied force is indicative of cellular avidity, i.e. the larger the percentage of cells that is bound the higher the cellular avidity, it is useful to refer to the percentage cells. Of course, one may use a different measure which relates to cellular avidity. One may also refer to the percentage of detached cells instead, wherein conversely a low number is indicative of a relative higher cellular avidity. Instead of percentage, one may also provide the ratio of cells that remain attached divided by the total number of cells that interacted, or provide the ratio for detached cells. One may also, in case of a cellular avidity plot as shown herein determine the area under (or above) the curve. One may also, in case a fixed number of cells is to be provided to a target surface, simply provide the number of cells that have detached and/or remained attached. In any case, as long as a unit is provided that is representative of the number of cells that have detached or cells that have remained attached, relative to the total number of cells that have interacted, such a unit may be contemplated. Such a unit allows for ranking cellular avidities, when comparing e.g. different cells of interest. Providing such a unit may be referred to as providing a cellular avidity score.

Hence, instead of determining the percentage of cells that remain attached to the target cells, relative to the cells of the contacting step, in the means and methods any unit may be provided that is representative of the number of cells that have detached or cells that have remained attached, relative to the total number of cells that have interacted, as such a unit can be regarded as a unit of cellular avidity. A preferred unit of cellular avidity, to which also may be referred to as a cellular avidity score, may be the percentage of cells that remains attached, relative to the cells that have interacted, the latter being set at 100%. Hence, in determining cellular avidity highly preferably, a cellular avidity score is provided.

Any suitable force application method may be contemplated in accordance with the invention. Increasing the force can be well controlled with acceleration based methods of applying force such as centrifugation, with shear flow and with acoustic force, which are all suitable means to be used in the methods in accordance with the invention but any other means of controllably causing a force on the cells attached to the target cells thereby forcing them away from the target cells, may be contemplated. In a further embodiment, the applied force is a force ramp, preferably a linear force ramp. It is understood that when a force is selected to be applied it can be a constant force applied for a defined period. The forces applied may be in various forms as a function of time. Preferably however, the applied force is an increasing force, that is, after the incubation step, an increasing force is applied for a defined period until a defined end force is reached. For example, as shown in the example section, in 150 seconds, a linear force ramp is applied resulting in a defined end force of 1000 pN.

With regard to the cellular avidity, it is understood that this is to express the strength of binding to the target cells.

In a further embodiment, the ratio or difference between the cellular avidity of cells of interest and control cells is determined. As the aim of using the blocking reagent like the NHS reagent is to reduce background binding, the ratio of the cellular avidity between cells of interest and control cells is preferably to be as large as possible. Ideally, control cell background binding is about 15% or less and cells of interest binding is about 85-95%. Hence, calculating the ratio or difference may be of interest when e.g. different conditions are tested in order to optimize background binding. Hence, it is understood that the means and methods in accordance with the invention also can comprise optimization of blocking conditions, which, once established can be further applied, e.g. in analyzing a variety of cells the cellular avidity. Hence, optimization of blocking conditions may be performed, such as i.a. shown herein. This way, optimal conditions utilizing a blocker as described herein can be established to provide e.g. for a percentage difference of at least 30%, or more between binding of control cells and cells of interest. As said, preferably, conditions may be selected with the largest difference between the percentages. Preferably, the percentage of control cells remaining attached is respectively 15% or less, and of cells of interest this preferably is in the range of 85% or higher. More preferably, in a scenario wherein e.g. conditions are to be selected for sorting purposes and the like, it may be desirable to have no control cells remaining attached, or at least a low percentage, such as 5% or less, 4% or less, or 3% or less. With any other unit or cellular avidity score determined, optimal differences can likewise be selected.

As said, with regard to the target cells and cells of interest (and control cells thereof) it is understood that this may involve cells that are to have a specific interaction, i.e. one cell carrying a receptor and the other cell having a ligand for the receptor. The term ligand and receptor in this sense and in accordance with the invention may define their inter-relationship. The term receptor may not be construed to be limiting in any way and is understood to mean a protein presented at the cell surface which can (specifically) interact with another protein (ligand) presented at a another cell. The terms ligand and receptor are used to indicate a complementarity which is important for specific recognition between cells without restrictions on the complementary molecules that can be contemplated. For example, one cell may have a T cell receptor or a CAR-T, and the other cell may present an antigen for the T-cell receptor, e.g. presenting an antigen via MHC. Such interactions are of interest to study and to modify. Hence, such cells of interest, and control cell thereof not carrying e.g. the CAR-T, and target cells are in particular of use in the means and methods of the invention, as exemplified in the example section herein. In a preferred embodiment immune effector cells are provided as cells of interest, and target cells expressing an antigen which the cell of interest is to specifically target. Receptors that may be of interest in accordance with the invention is a CAR, a TCR, a stimulatory or inhibitory coreceptor, or a receptor engaged via a bispecific antibody. For example, effector cells may be selected from T cells, NK cells, dendritic cells, macrophages, or monocytes. Such cells may be genetically modified, e.g. provided with e.g. a CAR. As said, target cells that are cancer cells, or other cells expressing an antigen, e.g. viral antigens or the like.

Advantageously, of a variety of cells of interest the cellular avidity may be determined. This is for example of interest when it is desirable to provide for a candidate CAR, a variety of cellular avidities. By providing a variety of CARs, candidates can be selected and subsequently assessed with regard to functionality, e.g. by in vivo experiments. As cellular avidity is an important parameter for function, this way highly efficiently, suitable candidates can be selected. Accordingly, in a further embodiment, of a variety of cells of interest the cellular avidity is determined.

Of course, the target cells attached to a surface that have been treated with the blocking agent, e.g. the NHS reagent, are not only useful for determining cellular avidity, but can also be utilized in further methods focusing e.g. on other aspects in which cellular avidity can be involved. For example, further methods can include methods for identifying a candidate agent capable of modulating the cellular avidity between a cell of interest and a target cell, wherein in the incubation step, and cellular avidity determination step of the method as defined above agent(s) are provided and included in these steps. This way, when it is for instance of importance to block or enhance a particular interaction, agent(s) capable of doing so can be identified. Furthermore, the target cells attached to a surface as prepared in accordance with the invention can also be used in methods for screening of different cells of interest, by performing the method as described above, optionally in the presence of agent(s) as defined above, and comparing determined cellular avidities for the different cells of interest. This way, for example, in case agents are present that are known to modulate a desired interaction between a cell of interest and a target cell, receptor/ligand interactions may be selected that are not affected by such agents, or, conversely, are aided by such agents. Hence, in one embodiment, a candidate agent is provided for modulating the avidity between a cell of interest and a target cell, and wherein said agent is present in the incubation step and step of determining cellular avidity.

Of course, in case target cells attached to a surface as prepared in accordance with the invention are provided, with an optional modulating agent, and shown to provide for selectivity with regard to binding of cells of interest to the target cells, as opposed to control cells, such can also be used for selecting and/or sorting cells of interest. Hence, in another embodiment, a method is provided for selecting and/or sorting cells of interest, comprising the steps of the method as defined above, and optionally in the presence of agent(s) as defined above, comprising the further step of applying the selected force on the cells of interest and subsequently selecting and/or sorting cells of interest that have detached and/or that remain attached to the target cells. Hence in another embodiment, a method is comprising the steps of the method of determining cellular avidity as described above, wherein the method is for use in sorting and/or screening of cells of interest.

Accordingly, a method is provided sorting and/or screening of cells of interest comprising the steps of:
providing target cells attached to a coated surface as prepared in accordance with the invention,
providing cells of interest;
incubating the cells of interest with the target cells attached to the surface;
applying a force on the cells of interest
selecting and/or sorting cells of interest that have detached and/or that remain attached to the target cells.

Cells of interest which can be selected and/or sorted and thus be obtained accordingly with means and methods in accordance with the invention may subsequently in a final step be admixed with a pharmaceutically acceptable buffer or otherwise pharmaceutical acceptably formulated.

In a further embodiment, methods may be of use for the screening of candidate agents as well. Hence, a further method is provided for screening candidate agents for modulating cellular avidity between of cells of interest and target cells comprising the steps of:
providing target cells attached to a coated surface as prepared in accordance with the invention,
providing cells of interest;
incubating the cells of interest with the target cells attached to the surface;
applying a force on the cells of interest
determining cells of interest that have detached and/or that remain attached to the target cells and provide a cellular avidity score.

In another embodiment, the use is provided of an NHS ester reagent in a blocking reaction of cells attached to a coated surface, wherein said surface was coated with a polypeptide. Hence, a use of providing of an NHS ester reagent for any of the means and methods as disclosed herein.

In another embodiment, target cells attached to a surface coated with a polypeptide are provided, wherein said target cells attached to the surface have been treated with an NHS ester reagent. Preferably, suitable surfaces and/or NHS ester reagents can be selected such as described herein.

In a further embodiment, a kit of parts is provided comprising a surface coated with a polypeptide for attachment of target cells and an NHS ester reagent. In a further embodiment, suitable coated surfaces and/or NHS ester reagents can be selected such as described herein. Moreover, instructions can be provided in such a kit of parts for contacting the target cells on the coated surface to allow the target cells to attach to the coated surface, and for blocking the target cells attached to the coated surface with the NHS ester agent. Such instructions are preferably in accordance with the means and methods of the invention as described herein.

In yet another further embodiment, a kit of parts is provided comprising a surface for attachment of target cells, a coating composition comprising a polypeptide, and an NHS ester reagent. In a further embodiment, coating composition comprising a polypeptide, suitable surfaces and/or NHS ester reagents can be selected such as described herein. Such a kit of parts may comprise further instructions for coating the surface with a polypeptide to provide a coated surface, contacting the target cells on the coated surface to allow the target cells to attach to the coated surface, and for blocking the target cells attached to the coated surface with the NHS ester agent. Such instructions are preferably in accordance with the means and methods of the invention as described herein.

In another embodiment, a cell engager may be provided. Cell engagers include antibodies, or the like, which are capable of binding to a target cell and a cell of interest, e.g. an effector cell. Such antibodies may include single chain antibodies comprising two binding domains such as scFv domain, and include BiTEs (i.e. bispecific T cell engagers) or the like. A conventional antibody design includes heavy and light chains, with one half of the antibody (one heavy chain and one light chain) engaging with a target cell, and the other half of the antibody (another heavy chain and another light chain) engaging with an effector cell, wherein preferably, the Fc domain is made inert. In any case, suitable cell engagers are widely known in the art and the current invention allows to study and/or determine cellular avidity, e.g. synapse formation, of induced by a cell engager between a target cell and an effector cell. Hence, accordingly, in the means and methods in accordance with the invention, a cell engager may be provided in addition, and e.g. the cellular avidity score is determined, induced by said cell engager between a cell of interest (e.g. an effector cell) and a target cell, said cell engager having a binding region capable of binding the cell of interest (e.g. an effector cell) and a binding region capable of binding the target cell. It is understood that the incubation step in the methods of the invention can thus be performed in the presence of the cell engager to allow the cells to interact, i.e. effector cells and target cells, and form a bond, e.g. a synapse, via the cell engager.

Accordingly, in one embodiment, in methods in accordance with the invention, a cell engager is provided capable of binding an effector cell and the target cell and inducing synapse formation, and the cell engager is included in the incubation step. Furthermore, such methods are highly useful for screening cell engagers, e.g. by identifying cell engagers that are particular capable of inducing a synapse.

It is understood that a synapse is a specialized structure that forms when the plasma membranes of two cells come into close proximity to transmit signals. Synapses can form between cells of interest and target cells, when e.g. the cells of interest are effector cells. Cells of the immune system form synapses that are essential for cell activation and function. Lymphocytes such as T cells, B cells and natural killer (NK) cells form synapses that can be referred to as immunological synapses. Such a synapse typically forms between effector cells and target cells, e.g. cells presenting an antigen. A non-limiting example is e.g. a T cell or a CAR-T cell and a cancer cell. The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses.

In the case of a T cell, a synapse can be formed between the lymphocyte and antigen-presenting cells (APCs) during the recognition of the peptide antigen-major histocompatibility complex (pMHC) ligand by the T-cell antigen receptor (TCR). The TCR and pMHC are both membrane-bound so the TCR will only be triggered by its ligand at the interface between T cells and APCs. A synapse can be observed at the T cell-APC interface as concentric rings by confocal microscopy, often referred to as “bull's eye” (Huppa, J. B., & Davis, M. M. (2003). T-cell-antigen recognition and the immunological synapse. Nature Reviews Immunology, 3(12), 973-983). These rings were named the central, peripheral, and distal supramolecular activation cluster (i.e. respectively cSMAC, pSMAC and dSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this ringed structure (“bull's eye”) is however not universal and other formations such as “multifocal immunological synapses” between T cells and dendritic cells, or the like, have been described.

The immunological synapse can be considered to be any structure formed at the interface resulting from a functional and specific effector-target cell interaction, such as for example T-cell-APC contacts. Markers associated with effector cells and synapse formation include one or more of CD43, CD44, CD45, LFA-1, Talin, F-actin, ZAP70, CD2, CD4, CD8, CD3, CD28, PD-1, ICOS, and TCR. Markers of target cells include one or more of ICAM-1 (associates with LFA-1), CD48/58 (interacting with CD2) CD80/CD86 (interacting with CTLA-4 and CD28), PDL1/PDL2 (associating with PD-1), and MHC presenting the antigen (that specifically interacts with the TCR). These markers, and concentrations thereof, may be detected e.g. with fluorescent labels at the interface between effector cell and target cell, or intracellularly in close proximity to the synaptic interface.

For example, markers of effector cells that have been associated with dSMAC are CD43, CD44, CD45. The effector cell markers LFA-1, Talin, F-actin, CD2, CD4 and CD8 have been associated with pSMAC. The markers CD3, CD28, PD-1, ICOS, and TCR of effector cells have been associated with cSMAC. Markers that have been associated with a synapse on a target cell are ICAM-1 (associates with LFA-1), CD48/58 (interacting with CD2) CD80/CD86 (interacting with CTLA-4 and CD28), and PDL1/PDL2 (associating with PD-1), and of course an MHC presenting the antigen (that specifically interacts with the TCR). These markers have been shown to be associated with synapses in a TCR-MHC interaction. Likewise, cell engagers (such as a bispecific antibody that e.g. binds CD3 on an effector cell with one arm and with the other arm an antigen on a target cell) which engage an effector cell with a target cell, can trigger the formation of a similar synapse structure as observed with a classic TCR-MHC interaction.

With regard to CARs of e.g. CAR T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 ζ-chain. Later generations combined intracellular signalling domains from various costimulatory protein receptors (e.g., CD28, 41BB, ICOS) incorporated in the cytoplasmic tail of the CAR to enhance signalling further. Further generations also incorporated in their design an inducible release of transgenic immune modifiers, such as IL-12, to shape the tumor environment by augmenting e.g. T-cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins. Synapses formed between CARs and target cells may not provide a classical bull's-eye like structure with a well-characterized SMAC domain, but may result in less organized pattern. Multiple CAR microclusters form and signalling molecules, which are dispersed in the center of the synapse interface.

Synapse formation is a spatiotemporal process that starts e.g. by TCR binding to MHC or binding of an antigen with CAR or cell engagement, and subsequent phosphorylation of the cytosolic tails of CD3 resulting in a triggered state. This sets of a cascade of processes that result in an activated T cell state, which also depends on the effector cell type and its phenotypic state. Key processes include: calcium signalling, internal cell structure and/or cytoskeleton changes of effector cells, involving F-Actin, Talin, and changes in microtubules, centrosomes, lytic granules, nucleus position, and mitochondrial location. Transactivation of adhesion molecules, cytokine and marker expression. IFNγ, granzyme and perforin may be released by effector cells to thereby induce i.a. target cell killing. Also, in addition, in target cells apoptotic markers can be found, including ICAM-1 clustering, phosphatidyl translocation, mitochondrial depolarization, caspase-3 activation and DNA fragmentation. Hence, various stages of specific effector cell and target cell interaction and immune activation can be detected.

In any case, synapse formation, or a synapse can be determined by staining, i.e. with fluorescent labels, ligands, antibodies, or probes, or the like, which may be used on live cells or on fixed cells targeting the mechanisms involved in T cell activation and/or synapse formation. Sequencing based methods may also be used to identify activated T cells and thereby associate mRNA levels with the formation of stable synapses. T cell activation leads to changes in mRNA stability and expression. E.g. increases in expression of cytokine or secretory transcripts (IL2, IFNγ, granzyme, perforin) and proliferation pathways and either bulk or single-cell RNA sequencing may be used to detect these changes and correlate these to the number or synapse formed.

In any case, in the cells obtained, which can be either in the form of target cells bound to effector cells or separated cells, e.g. with trypsin, the marker associated with synapse formation can be determined. Thus, in a further embodiment, the marker associated with synapse formation is determined in either the target cell or the effector cell. In another embodiment, in the methods in accordance with the invention one or more markers associated with synapse formation are determined and the one or more markers are determined in the effector cells and/or in the target cells. When cells are labelled, either in a singlet state or doublet state, cells may be highly advantageously be sorted and collected and subsequently analysed.

In one embodiment, a method in accordance with the invention is provided, wherein the marker associated with synapse formation is selected from the group consisting of calcium signalling signatures; spatial clustering of synapse localized molecules such as LFA-1, CD28, CD3, Agrin; changes to internal cell structure and/or cytoskeleton such as F-Actin, Talin, microtubules, centrosome, lytic granules, nucleus position, mitochondrial relocation; changes in effector cell motility; changes in external cell morphology and/or cell shape; and apoptosis of target cells. Synapse formation includes the initiation of a synapse up to and including the establishment of a synapse in which, as described above but not necessarily limited thereto, markers associated with synapse formation are associated.

In a further embodiment, the marker for synapse formation is calcium signalling, which can be detected with fluorescent calcium indicators, such as Fura2 AM (available from Invitrogen, item nr. F1221), which marker is suitable for detection in effector cells and in live cells. Other suitable dyes to detect calcium signalling can be selected from the group of Fura Red AM, Indo-1, pentapotassium, Fluo-3, fluo-4, Calcium Green-1, Rhod-2 and X-Rhod-1, Oregon Green 488 BAPTA. Hence, in one embodiment, the marker for synapse formation is calcium signalling which is detected with an indicator selected from the group consisting of Fura2 AM, Fura Red AM, Indo-1, pentapotassium, Fluo-3, fluo-4, Calcium Green-1, Rhod-2 and X-Rhod-1, and Oregon Green 488 BAPTA. With these markers calcium signalling can be detected which is a hallmark of synapse formation.

Furthermore, membrane potential dyes may also be used a calcium signalling indicators, such as the Invitrogen FluoVolt™ Membrane Potential Kit (Catalog number: F10488). Depolarization of the synapse forming cells by using a slow-response potential-sensitive probe such as Invitrogen DiSBAC2(3) (Bis-(1,3-Diethylthiobarbituric Acid)Trimethine Oxonol), Catalog number: B413. Hence, in another embodiment the marker for synapse formation is detected utilizing membrane potential dyes.

In another embodiment the marker for synapse formation is cytoskeleton rearrangement, which can be detected in effector cells with live or fixed cells, using cell staining, e.g. F-actin can be detected with Phalloidin conjugates or CellMask™ (Invitrogen, item nr A57243). Other usable stains can include SiR-Actin, CellLight™ Talin-GFP, BacMam 2.0, or Tubulin Tracker Deep Red. Hence, in one embodiment, the marker for synapse formation is cytoskeleton rearrangement, which is detected with a stain selected from the group consisting of Phalloidin conjugates, CellMask™ SiR-Actin, Cell Light™ Talin-GFP, BacMam 2.0, or Tubulin Tracker Deep Red. With these markers, cytoskeleton rearrangement can be detected.

In another embodiment, the marker for synapse formation involves monitoring effector cell motility. Detection of effector cell motility can be performed by video/timelapse monitoring of effector cells that remain in contact with the target cells after the force has been exerted. Detection of synapse formation includes detecting effector cell immobility, i.e. upon synapse formation effector cells will stop moving and remain into contact with the target cell with which it forms a synapse. Cell motility can be detected by membrane staining of effector cells and imaging, or using brightfield, darkfield or phase contrast microscopy.

It is understood that for some of the methods for detecting a marker for synapse formation which use e.g. microscopy and monitoring of motility, or short-lived signals, may be combined with having cells provided with e.g. photoactivatable label, to, upon detection of the marker, activate such a label in the cell, such that in subsequent steps, e.g. sorting, FACS analysis or the like, cell for which the marker associated with synapse formation was detected can easily be tracked.

As described above, in accordance with the invention, markers associated with synapse formation can be determined, e.g. via utilizing labels or the like. However, it may be highly advantageous to sequence obtained cells and identify synapse formation by sequencing. It is understood that sequencing comprises nucleotide sequencing, e.g. sequencing of DNA and/or RNA as expressed in cells. Means and methods are widely known in the art to sequence DNA and/or RNA as expressed in the cell. Hence in another embodiment, in the methods in accordance with the invention the markers are determined with sequencing. This is in particular highly useful for such markers which are up- or downregulated in target cells and/or effector cells in forming a synapse or having a synapse. Suitable markers for T cell activation and synapse formation are transcripts linked to interferon expression, proliferation, and cytokine expression and include: Interferon pathway upregulation: CD4, IFIT3, IFIT2, STAT1, MX1, IRF7, ISG15, IFITM3, OAS2, JAK2, SOCS1, TRIM21; proliferation: LIF, IL2, CENPV, NME1, FABP5, ORC6, GOS2, GCK; cytokine expression: CCL3, IFNG, CCL4, XCL1, XCL2, CSF2, IL10, HOPX, TIM3, LAG3, PRF1, TNFRSF9, NKG7, IL26. Sequencing may also be used to identify non-synapse forming cells by detecting molecular signatures linked to resting cell states: FOXP3, CTLA4, MTNFRSF4, IRF4, BATF, TNFRSF18, TOX2, PRDMI, LEF1, ATM, SELL, KLF2, ITGA6, IL7R, CD52, S100A4, TGFB3, AQP3, NLRP3, KLF2, ITGB7.

It may be also advantageous to determine the molecular state of the target cells as this can give an indication of the cell killing potency of the effector cells. In the case of paired analysis were effector-target-cell doublets are recovered this enables direct linking of effector cell phenotype with their killing capabilities. Next to staining methods for apoptosis commonly used in the field sequencing or qPCR can be used to detect transcripts linked to apoptosis or cell survival in the target cell, markers include: Bcl-2 family (BCL-xL) caspases 3, caspases 7, cleaved PARP, bax, bad, bak, bid, puma noxa, bcl-2, bcl-xl, mcl-1, p53, and cytochrome c, Smac/Diablo, survivn, Mcl-1, RNA Y1.

As it is understood that changes in gene expression may take some time and are not necessarily immediately detectable, one may allow after the step of exerting the force, the cells to remain bound to the target cells for some time and have these remain attached to the surface as well. Alternatively, one may also separate e.g. doublets that remained after exerting the force and subsequently e.g. sort doublets in single wells, and allow these to remain for some time. A suitable time to allow for expression of markers associated with synapse formation may be from 1 hour to 24 hour.

This is highly advantageous as sequencing methods allow for single cell sequencing, which also allows for determining e.g. the sequences of receptors expressed by the cell as well. Hence, in a further embodiment, different receptors are determined and identified via sequencing. In yet another embodiment, sequencing comprises single cell sequencing. In still another embodiment, sequencing comprises sequencing the expressed genome. Sequencing the expressed genome is highly useful as it allows to determine up- and downregulated gene expression. Nevertheless, sequencing genomic DNA may be useful as it may also provide useful information e.g. epigenetic markers associated with in vivo potency and durable responses.

With regard to the methods for determining cellular avidity, e.g. in screening, or for determining e.g. a cellular avidity score, as described herein, in accordance with the invention, it is understood that this can take into account the number of cells that have remained bound to each other after applying the force on the cells of interest. In such methods, wherein the cells of interest are effector cells, advantageously, this may also take into account the number of cells that remained bound to each other having one or more markers associated with synapse formation. For example a cellular avidity score, or determining cellular avidity, which takes into account target cells and effector cells having a marker associated synapse formation may provide for a more accurate cellular avidity score which is more reflective of the function of e.g. effector cells, i.e. forming synapses with target cells. Hence, such a cellular avidity score may also be referred to as a functional cellular avidity score or a synaptic cellular avidity score, and it is understood that where herein cellular avidities or cellular avidity scores are determined in accordance with the invention, this may also include cellular avidity scores taking into account synapse markers. Hence, in further embodiments, synapse markers are used in the methods of the invention such as in screening of candidate agents, sorting and/or screening of cells of interest, i.e. of effector cells.

It was observed that when cells that remained bound and attached after a cellular avidity measurement were resuspended utilizing i.a. repeated pipetting and thus exerting a significant force, cells attached to the surface were detached and moreover, a portion of the cells that were bound to the attached cells became unbound. Hence, this implied that with such a process step a differential force can be applied on bound cells that can exceed the maximum force that is applied away from cells attached to a surface. This way, further cell-cell bonds that may be aspecific cell-cell bonds may thus be broken therewith providing substantially specific cell-cell bonds that remain, e.g. effector cells bound with target cells (and optionally a cell engager) that formed a synapse can be retained. Hence, in a further embodiment, after the force has been applied away from the attached cells, subsequently, the cells are resuspended and a differential force is applied such that substantially effector cells that are bound to each other via a synapse remain bound to each other and substantially cells that have formed aspecific bonds are unbound, i.e. have their cell-cell bonds broken. It is also understood that instead of applying the force away, the differential force may be applied instead, and that by applying a differential force, it may no longer be required to have cells attached to a surface, i.e. immobilized. Furthermore, it is also understood that after the differential force has been applied, cells are highly preferably separated and/or sorted in order to avoid further interaction between cells to avoid establishing additional cell-cell bonds. Hence, highly preferably, cells are collected and sorted and/or analysed after the differential force has been applied.

As is clear from the above, the type of force that is to be applied in accordance with the invention is a force capable of breaking cell-cell bonds, i.e. the force exerted causes cells bound to each other move away from each other to such an extent that a cell-cell bond may break or rupture. A differential force means that the force on one cell differs from the force on the other cell with regard to direction of the force and/or the magnitude of the force, resulting in a net force allowing to break cell-cell bonds if the differential force exceeds the binding force.

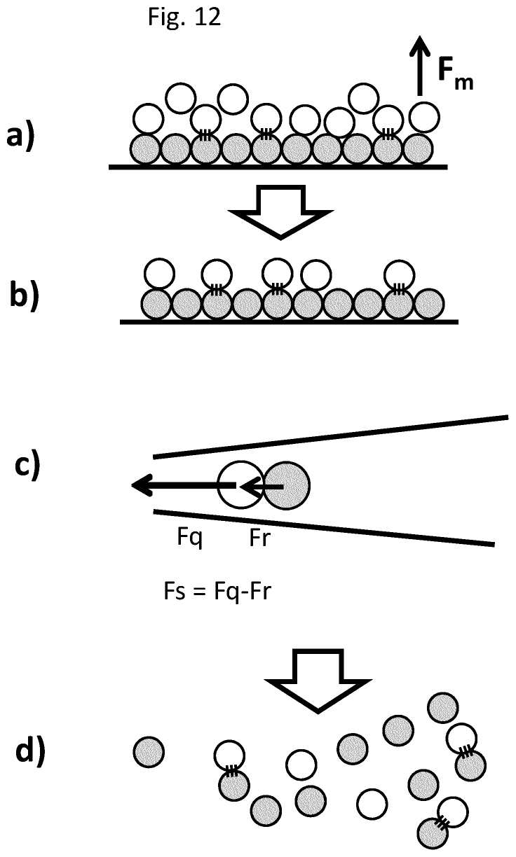

For example, when a target cell bound to an effector cell, a doublet, is forced through a nozzle, the closer to the throat of the nozzle the faster the flow. This means that the first cell to enter the nozzle is subjected to a stronger acceleration than the cell lagging behind and the cells experience a differential force resulting in a net force which can result in cell-cell bond rupture, provided the force is large enough (see e.g. FIG. 12, in particular 12c). A differential force that can be applied includes a shear force, e.g. such as can be applied utilizing repeated pipetting (repeated upwards and downwards flow of the sample) or flow through a nozzle.

Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J Histochem Cytochem. 1979, 27 (1), 635-641). Also, by properly tuning the forces in a flow-cytometer normally used to measure cell deformations, such as e.g. described in Otto, et al. Nat Methods 12, 199-202 (2015) suitable forces can be applied. Tuning can be achieved e.g. by changing the nozzle size or geometry and/or the flow speeds used. Other suitable devices known in the art may include for a vortex mixer, with which shear forces may be suitably applied as well. Accordingly, in one embodiment, the force applied involves a shear force.

In another embodiment, the force applied is an ultrasonic force. It is understood, as outline above, that such ultrasonic forces are not forces such as applied e.g. in a device as available from LUMICKS, wherein the force is away from attached cells (e.g. such as in the LUMICKS z-Movi® Cell Avidity Analyzer, e.g. as used by Larson et al., Nature 604)7906):1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter 11.2.8, page 576; and Moore et al., Experimental Cell Research, Volume 65, Issue 1, 1971, Pages 228-232).

In any case, suitable applied forces which are known in the art include e.g. a force in the range of 50 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 50 pN, or at least 100 pN, or at least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 100 pN-10 nN, from 500 pN-10 nN, from 1 nN-10 nN. In still a further embodiment, the force is selected from the range of 500 pN-5 nN, from 500 pN-4 pN, from 500 pN-3 pN. For example, a suitable amount of force that can be exerted between cells (e.g. such as in the z-Movi® device) can be selected to be in the range of 200 pN-3000 pN. Of course, these force ranges are known to be useful with cells attached to a surface, and the maximum force that may be selected may exceed 3000 pN as it is not required to have the cells to remain attached to a surface in accordance with the invention.

Accordingly, it is understood that in these embodiments, the differential force to be applied does not require either of the target cells or cells of interest, e.g. effector cells, to be or remain to be attached, and the differential force is a force selected from the range of 50 pN-10 nN. In another embodiment, in methods in accordance with the invention wherein the force that is applied is a differential force, neither the target cells nor the cells of interest, e.g. effector cells, require to remain to be attached to a surface, and the differential force is applied in the range of 50 pN-10 nN, thereby providing cells substantially comprised of target cells, cells of interest, and cells of interest bound to target cells. It is understood that by selecting an appropriate force, aspecific cell-cell bonds may be selectively broken, while retaining specific cell-cell bonds between cells of interest and target cells. For example, in case of effector cells, effector cells bound with target cells via a synapse may be retained.

Without being bound by theory, as is understood the range of force that may break an aspecific cell-cell bond versus a specific cell-cell bond that forms a synapse differs. This difference can be to such an extent that the ranges of the required forces do not overlap. It is understood that some overlap may occur. Hence the force that is selected, as outline above, may allow for aspecific cell-cell bonds remaining and some specific cell-cell bonds that formed a synapse to break. In case there is substantially no overlap, a differential force can be selected, as outline above, which allows substantially for aspecific cell-cell bonds to break, while substantially retaining specific cell-cell bonds that formed a synapse. In case there is no overlap, and ranges are sufficiently far apart, a differential force may be selected, as outline above, which allows for aspecific cell-cell bonds to break while retaining specific cell-cell bonds that formed a synapse. As also outlined herein, the portion of cell-cell bonds that remains after exerting a force and has a synapse, can be determined by determining the presence or absence of a marker associated with synapse formation. Likewise the same principles can be applied for further cells of interest and target cells.

In one embodiment, after the step of applying a force in the methods of the invention, the cells are resuspended and a subsequent differential force is applied such that formed aspecific cell-cell bonds are broken. In another embodiment, in methods of the invention either the target cells or cells of interest (e.g. effector cells) are attached to a surface, and, in the step of applying the force instead a differential force is applied. In yet another embodiment, in methods of the invention wherein the cells of interest are effector cells, either the target cells or effector cells are attached to a surface, and, in the step of applying the force instead a differential force is applied, wherein the differential force is such that cells that are bound via a synapse remain bound to each other and formed aspecific cell-cell bonds are broken.

### EXAMPLES

### Example 1

### Materials and Methods

### Primary Cells and Cell Lines

Untransduced or FMC63-transduced primary CAR T cells were obtained from ProMab Biotechnologies Inc. (Anti-CD19-TF-CD28-CD3z (PMC-152)) propagated with CD3/CD28 dynabeads (Thermo Fisher Scientific) and 90 IU/mL of IL-2 (Miltenyi Biotec), or Untransduced or FMC63 CAR transduced Jurkat were used (Creative Biolabs).

Raji cells or Hela-CD19 cells (ProMab Biotechnologies Inc., PM-HELA-CD19) were used as target cells. Primary CAR-T cells were cultured in RPMI+Glutamax supplemented with 50 μM β-Mercaptoethanol, 5% heat inactivated human serum, and Penicillin-Streptomycin. Jurkat and target cells were cultured with RPMI+Glutamax supplemented with 10% heat inactivated fetal bovine serum and Penicillin-Streptomycin. Hela-CD19 were cultured with DMEM+Glutamax supplemented with 10% heat inactivated fetal bovine serum and Penicillin-Streptomycin.

### Avidity Measurement

z-Movi® chips (obtained and as available from LUMICKS (<https://lumicks.com/products/z-movi-cell-interaction-studies/>) were coated with Poly-L-Lysine (#P4707-50ML, Sigma) diluted 1:5 in PBS for 10 minutes at room temperature, or with human Fibronectin (#F0895, Sigma) at 0.1 mg/mL overnight at room temperature. The following day the target cells were seeded in serum free RPMI medium, incubated 1 h, and subsequently the medium replaced with complete RPMI medium. The chips were incubated for an additional hour before the blocking reaction. The effector cells were stained with CellTrace™ Far Red dye (Thermo Fisher Scientific) at 1 μM for 15 minutes in PBS at 37° C., then resuspended at 10 million/mL in complete medium and used for the avidity experiments. The stained effector cells (i.e. cells of interest) were introduced in the target cell-seeded flow cell, incubated for 5 minutes, and then a 1 to 1000 pN force ramp was applied throughout 2.5 minutes using z-Movi® operated with the Oceon V1.2 software. At the end of the avidity measurements, the monolayer was stained with a Trypan Blue solution to qualitatively assess the target cell viability. The avidity experiment analysis was performed using Oceon V1.2.

### Blocking

The MS(PEG)₄ or BS(PEG)₅ (Thermo Fisher Scientific) (see FIG. 2) were dissolved in anhydrous DMSO (Merck) then diluted in PBS to obtain a final concentration of 5 mM or as otherwise indicated. The target cell-seeded chips (or coated only with PLL or fibronectin) were rinsed with PBS before introducing the blocking solution (or a PBS solution with DMSO as control sample). The chips were incubated for 15 minutes at room temperature and the reaction was stopped with TBS (50 mM Tris pH7.4, 150 mM NaCl). The chips were subsequently equilibrated with complete medium and used for avidity measurements. Furthermore, blocking was also tested with incubations of 1 hour with serum free RPMI medium, 20% fetal bovine serum (FBS), 50% FBS, 50% human serum (HS), 10% bovine serum albumin (BSA), NALM6 condition medium.

### Functional Assays

Target cells were treated with DMSO or blocking agent using the protocol above, resuspended at 1 million/mL in RPMI complete medium and mixed at 1:1 ratio with the indicated effector cells. For the IFNγ release assay the samples were incubated for 7 h in the incubator before spinning down and use of the supernatant to perform the ELISA (Biolegend, ELISA MAX™ Deluxe Set Human IFNγ). For the CD69 assay, the target and effector cells prepared as described above were co-incubated for 5 h in the incubator before being stained using anti-CD3 and anti-CD69 antibodies (clone SK7 and FN50, Biolegend).

### Results

As the Effector cell-coating matrix interaction can contribute to background binding we aimed to test different conditions (blocking agents) that may reduce the interaction between effector cell and the coating matrix to ultimately reduce background binding in avidity experiments. We coated z-Movi® chips with Poly-L-Lysine (PLL) or human fibronectin and performed different blocking treatments before introducing the Jurkat effector cells and evaluation of the fraction of cells attached to the glass after application of the acoustic force.

The irreversible neutralization of the lysine side chains on PLL and fibronectin using an N-Hydroxysuccinimide (NHS)-based blocking agent (BS(PEG)₅) reduced the binding of the effector cells to the coating matrix (FIG. 4). The effect was less prominent on fibronectin (FIG. 5), which can be explained as some of the domains engaged by the effector cell on this molecule are lysine-free and may be less affected by the blocking agent. BS(PEG)₅ resulted in both instances in the least amount of Jurkat cells bound.

We next validated the use of NHS-based blockers to reduce background binding of the effector cells in the presence of the monolayer. We used as cells of interest a Jurkat cell line transduced to express the FMC63 Chimeric Antigen Receptor (CAR) or untransduced (UNT) as control cells. As target cells we used the Raji cell line, endogenously expressing the CD19 antigen, seeded on PLL coated chips.

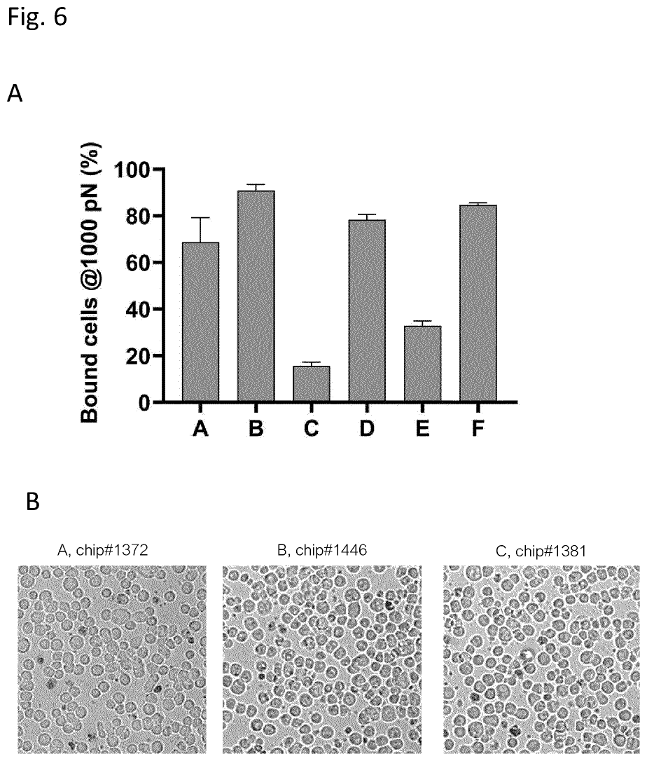

Reacting the Raji-seeded chips with blocking agent carrying one (MS(PEG)₄) or two (BS(PEG)₅) NHS groups before performing the avidity measurements was effective in reducing the background binding of the UNT sample (reducing cells bound from about 50% to about 20%) and preserved, to an extent, the binding of the CAR sample (from about 80% to about 70%), see FIG. 6A. Importantly, the assessment of the target cell viability with Trypan Blue staining at the end of the avidity measurement did not show differences with regard to cell staining and cell morphology between the non-blocked and blocked chips indicating that a decrease in observed avidity after blocking cannot be attributed to a decrease in monolayer viability (FIG. 6B).

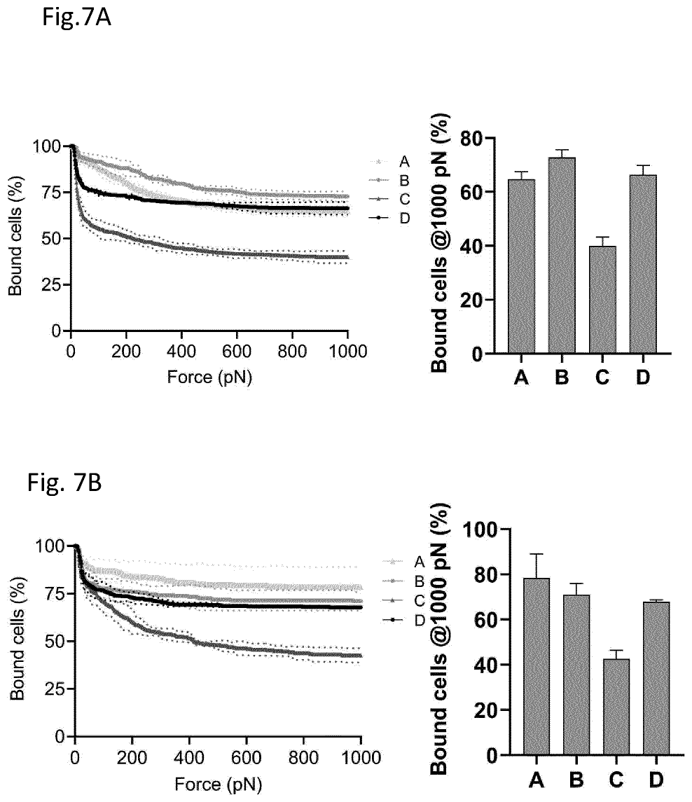

We then performed the experiments with the blocking agents with primary T cells expressing a FMC63 CAR (or not) layered on Raji cells. We observed that the observations made with Jurkat cells were recapitulated showing a considerable improvement of the negative/positive window on the chips subjected to the blocking reaction (FIG. 7).

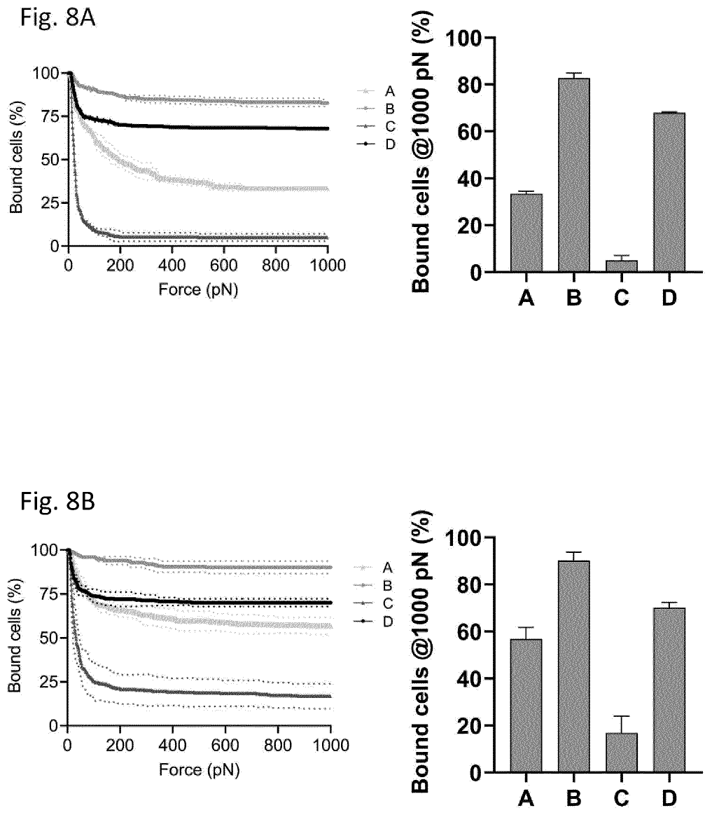

To confirm these findings with a different coating matrix and target cell line, we next tested the effect of the blocking reaction using a Hela cell line transduced to express CD19 and seeded on chips seeded with either PLL or fibronectin. We observed a substantial reduction of the background binding with a limited effect on the binding of the CAR-transduced sample during avidity (FIG. 8).

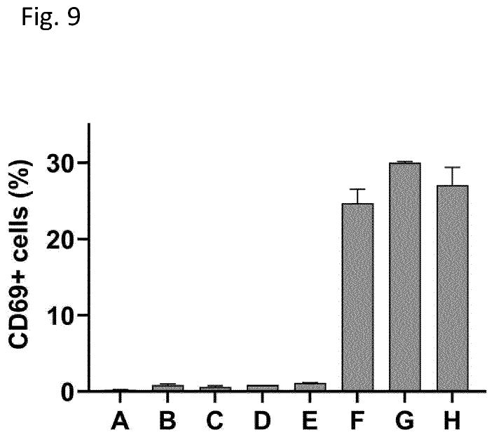

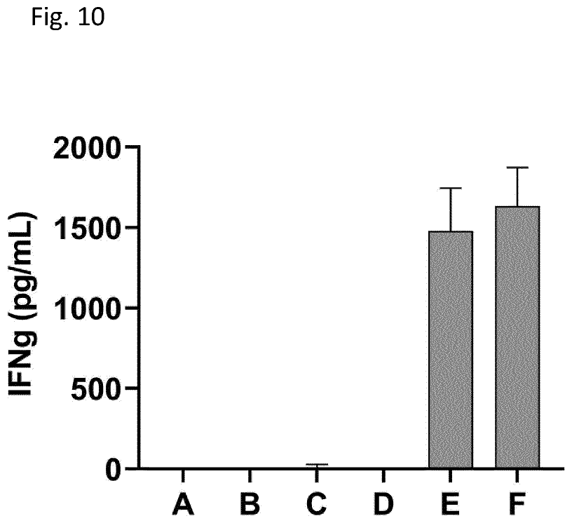

We next asked if reacting the target cells with NHS-based blockers could have an effect on the recognition of the target and consequently the activation of the effector cell. To test this, we performed functional assays of both primary T cells or Jurkat cells untransduced or transduced with FMC63 CAR, co-incubated with target cells that have been reacted or have not been reacted with the blocking agent. The upregulation of the CD69 activation marker in Jurkat (FIG. 9) and the IFNγ production on the CAR-transduced primary T cells (FIG. 10) was not apparently affected by the blocking of the target cells indicating that the CD19 target recognition by the CAR-T and subsequent activation of the CAR-T is not influenced by blocking the monolayer with NHS agents.

### Example 2

### Materials and Methods

### Primary Cells and Cell Lines

Untransduced or FMC63-transduced primary CAR T cells were obtained from ProMab Biotechnologies Inc. (Anti-CD19-TF-CD28-CD3z (PMC-152)) propagated with 15 CD3/CD28 dynabeads (Thermo Fisher Scientific) and 90 IU/mL of IL-2 (Miltenyi Biotec). Raji cells were used as target cells. Primary CAR-T cells were cultured in RPMI+Glutamax supplemented with 50 μM β-Mercaptoethanol, 5% heat inactivated human serum, and Penicillin-Streptomycin.

### Avidity Measurement

z-Movi® chips (obtained and as available from LUMICKS) were coated with Poly-L-Lysine (#P4707-50ML, Sigma) diluted 1:5 in PBS for 10 minutes at room temperature. The following day the target cells were seeded in PBS, incubated 0.75 h, and subsequently the monolayers were left untreated or reacted with BS(PEG)₉. For negative control the PLL coated chips were reacted with 5 mM MS(PEG)₄ before seeding the Raji cells. The effector cells were stained with CellTrace™ Far Red dye (Thermo Fisher 26 Scientific) at 1 μM for 15 minutes in PBS at 37° C., then resuspended at 10 million/mL in complete medium and used for the avidity experiments. The stained effector cells (i.e. cells of interest) were introduced in the target cell-seeded flow cell, incubated for 5 minutes, and then a 1 to 1000 pN force ramp was applied throughout 2.5 minutes using z-Movi® operated with the Oceon® V1.2 software. At the end of the avidity measurements, the monolayer was stained with a Trypan Blue solution to qualitatively assess the target cell viability. The avidity experiment analysis was performed using Oceon® V1.2.

### Crosslinking

The BS(PEG)₉ (Thermo Fisher Scientific) was dissolved in anhydrous DMSO (Merck) then diluted in PBS to obtain a final concentration of 10 mM. The target cell-seeded chips were rinsed with PBS before introducing the crosslinking solution (or a PBS 15 solution with DMSO as control sample). The chips were incubated for 15 minutes at room temperature and the reaction was stopped with complete RPMI medium. The chips were subsequently used for avidity measurements.

### Results

An important step in the avidity experiment workflow is the attachment of the target cell line on the glass surface of the z-Movi® chip. The use of polypeptides like Poly-L-Lysine as glass coating agents improves the attachment of the target cells to the surface, but many target cell lines do not adhere firmly to the PLL coated glass and can be lift off when the acoustic force is applied.

In order to overcome this limitation, it was reasoned that small molecules that could react both with the primary amine groups of the PLL and at the same time of the primary amines on the Lysine residues on surface cellular proteins could be used to stably link the target cell to the PLL coated glass. To test this hypothesis the NHS-based homo-bifunctional crosslinker BS(PEG)₉ was used.

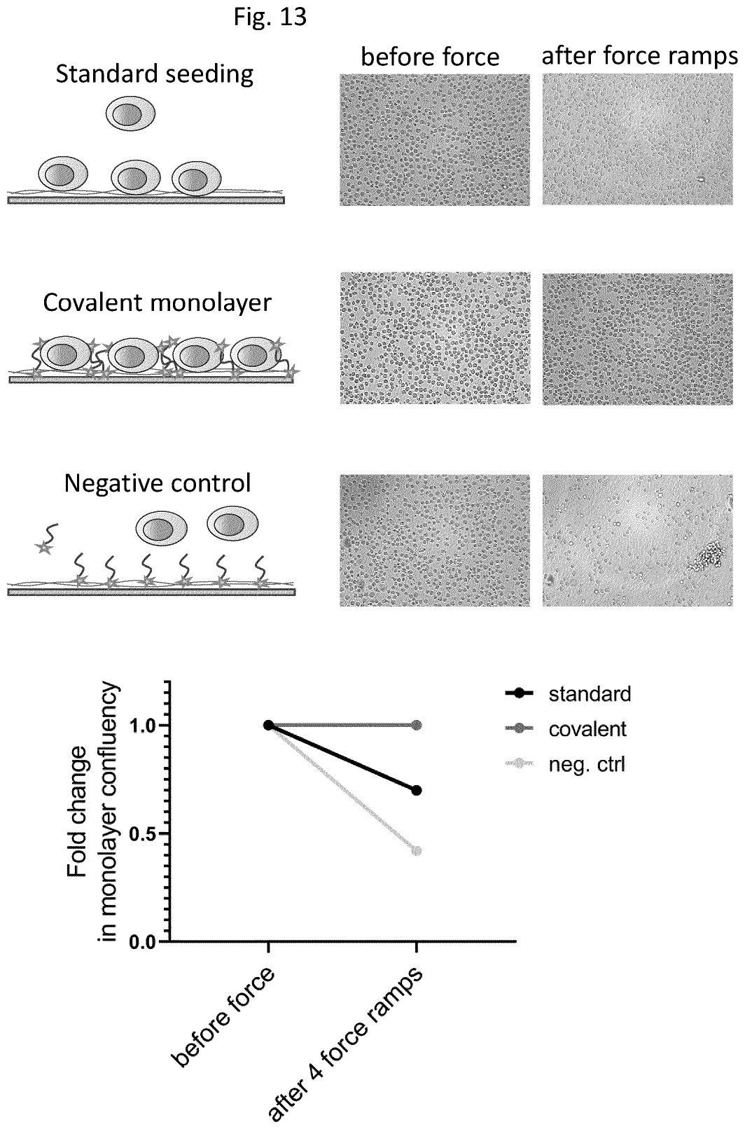

Raji cells were seeded on PLL coated chips with the standard procedure or performing a crosslinking reaction with BS(PEG)₉ after the initial seeding step and the monolayer robustness was tested by application of multiple acoustic force ramps. The chips seeded with the standard method displayed a progressive drop of monolayer confluency after force application, while the chips reacted with BS(PEG)₉ were not affected by the force (see FIG. 13). This result shows that BS(PEG)₉ stably connects the target cells to the PLL-coated chip. A chip reacted with the mono-functional crosslinker MS(PEG)₄ before the seeding of the Raji cells prevented the stable attachment of the target cells on the PLL-coated glass. It can be concluded that reacting target cell seeded PLL-coated chips with BS(PEG)₉ improves the stability of the target cell monolayer to acoustic force application.

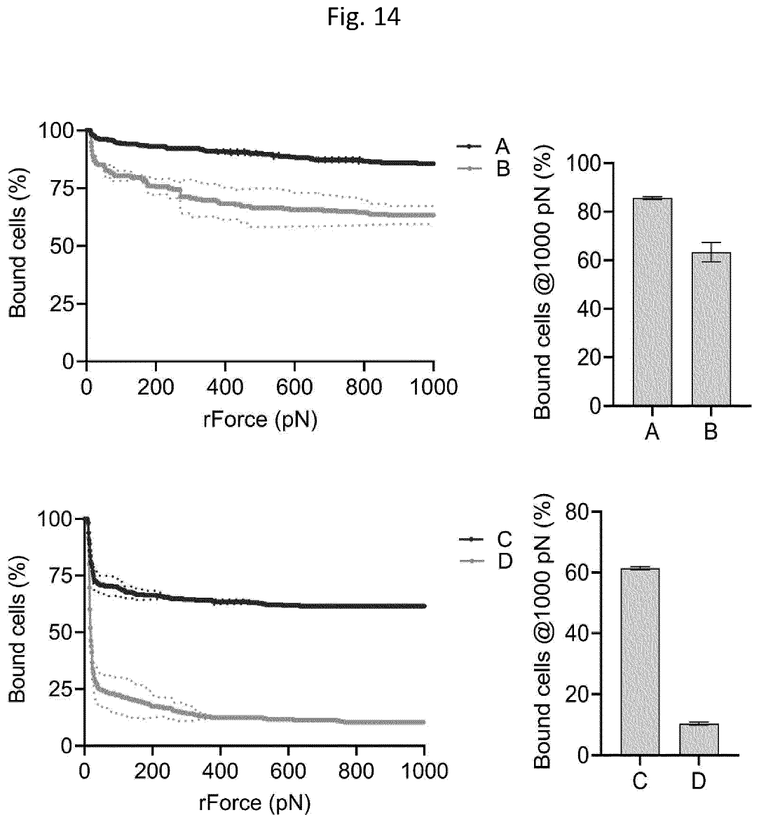

Next, the question was answered if chips seeded with the covalent monolayer method could allow the measurement of cell avidity when effector cells were introduced in the chip. When the avidity of FMC63-transduced primary T cells to target cells was compared with the standard and covalent monolayer seeding methods, the latter displayed a wider avidity window (see FIG. 14). The high aspecific binding observed with the standard seeding method was expected given the contribution of the free PLL to the binding of the effector cells. However, the reaction of the BS(PEG)₉ passivates the free PLL with a consequent improvement of the avidity window by reduction of the aspecific binding. It can be concluded that reacting target cell seeded PLL-coated chips with BS(PEG)₉ reduces the aspecific binding of primary control T cells and improves the positive/negative window in avidity experiments.

1. A method for preparing target cells attached to a coated surface, comprising the steps of:
providing target cells attached to a surface coated with a polypeptide;
providing an NHS ester reagent;
blocking the target cells attached to the coated surface with the NHS ester agent.

2. A method for preparing target cells attached to a coated surface, comprising the steps of:
providing a surface coated with a polypeptide for attachment of target cells;
providing target cells;
providing an NHS ester reagent;
contacting the target cells with the coated surface and allowing the target cells to attach to the coated surface;
blocking the coated surface with attached target cells with the NHS ester agent.

3. A method for preparing target cells attached to a coated surface, comprising the steps of:
providing a surface for attachment of target cells;
providing target cells;
provide a polypeptide for coating;
providing an NHS ester reagent;
coating the surface with a polypeptide to provide a coated surface;
contacting the target cells on the coated surface and allowing the target cells to attach to the coated surface;
blocking the target cells attached to the coated surface with the NHS ester agent.

4. The method in accordance with claim 1, wherein the NHS ester agent is a PEG-NHS ester.

5. The method in accordance with claim 4, wherein the PEG-NHS ester agent is a BS(PEG)n or MS(PEG)n.

6. The method in accordance with claim 5, wherein BS(PEG)n is BS(PEG)₅ or BS(PEG)₉ or wherein MS(PEG)n is MS(PEG)₄.

7. The method in accordance with claim 1, wherein the coating is selected from the group consisting of fibronectin, poly-L-lysine, poly-D-lysine, poly-L-ornithine, laminin, collagen, fibronectin, fibrinogen, vitronectin, and osteopontin.

8. The method in accordance with claim 1, wherein the provided surface for attachment is a glass surface, preferably a glass surface in a chip.

9. A method for determining cellular avidity comprising:
providing target cells attached to a coated surface as defined in claim 1,
providing cells of interest;
incubating the target cells attached to the coated surface with the provided cells of interest; and
determining the cellular avidity of the cells of interest with the target cells.

10. The method in accordance with claim 9, wherein control cells of the target cells or the cells of interest are provided and the cellular avidity determined thereof as well.

11. The method in accordance with claim 9, wherein the cellular avidity is determined by exerting an acoustic force, shear flow or a centrifugal force.

12. The method in accordance with claim 9, wherein a candidate agent is provided for modulating the cellular avidity between a cell of interest and a target cell, and wherein said agent is present in the incubation step.

13. The method comprising the steps in accordance with claim 12, for the screening of candidate agents.

14. Method for sorting and/or screening of cells of interest comprising the steps of:
providing target cells attached to a coated surface as defined in claim 1,
providing cells of interest;
incubating the cells of interest with the target cells attached to the surface;
applying a force on the cells of interest
selecting and/or sorting cells of interest that have detached and/or that remain attached to the target cells.

15. Method in accordance with claim 9, wherein the cells of interest are effector cells.

16. Use of an NHS ester reagent in a blocking reaction in a method in accordance with claim 1.
